(19) United States
(12) Patent Application Publication
(10) Pub. No.: US 2025/0375959 A1
(43) Pub. Date: Dec. 11, 2025

(54) MULTILAYER, FLEXIBLE, POROUS, DISSOLVABLE SOLID SHEET ARTICLE

(71) Applicant: The Procter & Gamble Company, Cincinnati, OH (US)

(72) Inventors: Carl David MAC NAMARA, Beijing (CN); Hong Sing TAN, Beijing (CN); Xiaobin CHU, Beijing (CN)

(21) Appl. No.: 19/229,230

(22) Filed: Jun. 5, 2025

(30) Foreign Application Priority Data
Jun. 6, 2024 (WO) PCT/CN2024/097810

(51) Int. Cl. B32B 27/30; B32B 27/20; B32B 27/22

(52) U.S. Cl. CPC B32B 27/306; B32B 27/20; B32B 27/22; B32B 2250/02; B32B 2266/06; B32B 2266/10; B32B 2270/00; B32B 2305/022; B32B 2305/54; B32B 2307/7166; B32B 2307/72; B32B 2307/737; B32B 2307/7376; B32B 2315/00; B32B 2329/04; B32B 2379/00

(57) ABSTRACT

The present invention relates to a multilayer, flexible, porous, dissolvable solid sheet article.

### FIELD OF THE INVENTION

The present invention relates to a multilayer, flexible, porous, dissolvable solid sheet article.

### BACKGROUND OF THE INVENTION

Flexible dissolvable solid sheets comprising surfactant(s) and/or other active ingredients in a water-soluble polymeric carrier or matrix are well known. Such sheets are particularly useful for delivering surfactants and/or other active ingredients upon dissolution in water. In comparison with traditional granular or liquid forms in the same product category, such sheets have better structural integrity, are more concentrated and easier to store, ship/transport, carry, and handle. In comparison with the solid tablet form in the same product category, such sheets are more flexible and less brittle, with better sensory appeal to the consumers.

To make such flexible dissolvable solid sheets, both a drum-based batch process and a belt-based continuous process have been developed. Particularly, a drying step is included in these manufacturing processes of such flexible dissolvable sheets to form a wet pre-mixture into the solid sheets. Such drying step is usually through heating under an appropriate condition. Further, in a more preferred belt-based continuous process which has a much higher rate of manufacture compared to the drum-based batch process, the drying step may include multiple sub-steps with different settings of heating.

In order to provide a desirable porosity (so-called open cell structure), previous work has been attempted to optimize the drying step, including the optimization in the aspects of heating direction, heating temperature, heating duration, step-wised heating and the like. However, the manufacturing processes are still sensitive to the composition of the solid sheets probably because of inherent properties of the water-soluble polymers and surfactants. Especially, for multilayer solid sheet articles, it is even more challenging to provide a desirable porous solid structure (e.g. in the aspects of thickness, Percent Open Cell Content, pore size uniformity of pores and the like) together with an acceptable manufacturing process. Particularly, some formulae may not provide a desirable porous solid structure, e.g. high collapse % after drying; some formulae may cause manufacturing failures, e.g. over puffing during the drying step, being too sticky to be peeled off from the belt; and some formulae cannot provide a strong structure (e.g., may fall apart easily).

Further, in the aspect of surfactant system, alkyl alkoxy sulfates (AAS) and alkyl sulfates (AS) are commonly used in detergent products and provide good performance in cleaning. As such, a detergent sheet product containing AAS and/or AS is desirable.

Thus, a need still exists for an optimized composition of such solid sheet articles under certain circumstances (e.g., containing AAS and/or AS) which can work well in the current manufacturing process to provide a desired flexible, porous, dissolvable solid sheet article.

Additionally, a multilayer solid sheet article is desirable for consumers because it is more compact and appealing. However, the multilayer solid sheet article may have issues of delamination. As such, a need exists for an optimized composition of multilayer solid sheet articles which can overcome the issues of delamination.

### SUMMARY OF THE INVENTION

The present invention relates to a flexible, porous, dissolvable solid sheet article comprising a plurality of water-soluble sheets arranged in a stack, wherein each of the water-soluble sheets comprises a water-soluble polymer, a C₆-C₂₀ linear or branched alkylalkoxy sulfates (AAS) having a weight average degree of alkoxylation ranging from 0.5 to 10, and a C₆-C₂₀ linear or branched alkyl sulfates (AS), wherein said solid sheet article is characterized by: (i) a Percent Open Cell Content of from 80% to 100%; and (ii) an Overall Average Pore Size of from 100 μm to 2000 μm; wherein the weight ratio of said AAS over said AS is from 0.4 to 3.

In some embodiments, the weight ratio of said AAS over said AS is from 0.5 to 2, preferably from 0.7 to 1.5, more preferably from 0.8 to 1.3, most preferably from 0.9 to 1.1.

In some embodiments, each of the water-soluble sheets comprises less than 5%, preferably from 0 to 3%, more preferably from 0 to 1%, most preferably from 0 to 0.1%, of a non-ionic surfactant by total weight of the water-soluble sheet.

In some embodiments, said water-soluble polymer is selected from the group consisting of polyvinyl alcohols, polyvinylpyrrolidones, polyalkylene oxides, starch and starch derivatives, pullulan, gelatin, hydroxypropylmethylcelluloses, methycelluloses, carboxymethycelluloses, and any combinations thereof. Particularly, said water-soluble polymer has a weight average molecular weight of from 5,000 to 400,000 Daltons, more preferably from 10,000 to 300,000 Daltons, still more preferably from 15,000 to 200,000 Daltons, most preferably from 20,000 to 150,000 Daltons.

In some embodiments, the water-soluble polymer comprises a polyvinyl alcohol. Preferably, said solid sheet article comprises from 1% to 60%, preferably from 5% to 50%, more preferably from 10% to 40%, most preferably from 15% to 40%, of said polyvinyl alcohol by total weight of said solid sheet article.

In some embodiments, each of the water-soluble sheets comprises from 1% to 60%, preferably from 5% to 55%, more preferably from 10% to 53%, most preferably from 15% to 52%, of total surfactants by total weight of said solid sheet article.

In some embodiments, each of the water-soluble sheets comprises an additional surfactant, a perfume, a chelant, a polymer, a solvent, an antioxidant, or any combinations thereof.

In some embodiments, said additional surfactant is preferably selected from the group consisting of: a C₆-C₂₀ linear alkylbenzene sulfonate (LAS), a fatty acid, alkylamphoacetates and any combinations thereof; and/or

In some embodiments, said perfume is preferably selected from the group consisting of: free perfumes, perfume microcapsules, and any combinations thereof; and/or

In some embodiments, said chelant is preferably selected from the group consisting of diamine-N,N′-dipolyacid, monoamine monoamide-N,N′-dipolyacid, and N,N′-bis(2-hydroxybenzyl)ethylenediamine-N,N′-diacetic acid chelants (preferably EDDS (ethylenediaminedisuccinic acid)), carboxylic acids (preferably aminocarboxylic acids), phosphonic acids (preferably aminophosphonic acids) and polyphosphoric acids (in particular straight polyphosphoric acids), their salts and derivatives; and/or

In some embodiments, said polymer is preferably selected from the group consisting of polyalkylene imine polymer, polyalkylene oxide polymer and any combinations thereof; and/or

In some embodiments, said solvent is preferably selected from the group consisting of glycerol, propylene glycol, 1,3-propanediol, diethylene glycol, dipropylene glycol, ethanolamine, ethanol, water and any combinations thereof; and/or

In some embodiments, said antioxidant is preferably selected from the group consisting of C₁-C₂₂ linear alkyl esters of 3,5-bis(1,1-dimethylethyl)-4-hydroxy-benzenepropanoic acid and mixtures thereof.

In some embodiments, said solid sheet article is characterized by:
a Percent Open Cell Content of from 85% to 100%, preferably from 90% to 100%; and/or
an Overall Average Pore Size of from 150 μm to 1000 μm, preferably from 200 μm to 600 μm; and/or
an Average Cell Wall Thickness of from 5 μm to 200 μm, preferably from 10 μm to 100 μm, more preferably from 10 μm to 80 μm; and/or
a final moisture content of from 0.5% to 25%, preferably from 1% to 20%, more preferably from 3% to 15%, by weight of said solid sheet article; and/or.
a thickness of each sheet being from 0.5 mm to 4 mm, preferably from 0.7 mm to 3 mm, more preferably from 0.8 mm to 2 mm, most preferably from 1 mm to 1.5 mm; and/or
a basis weight of from 50 grams/m² to 500 grams/m², preferably from 80 grams/m² to 400 grams/m², more preferably from 100 grams/m² to 350 grams/m²; and/or
a density of from 0.05 grams/cm³ to 0.5 grams/cm³, preferably from 0.06 grams/cm³ to 0.4 grams/cm³, more preferably from 0.07 grams/cm³ to 0.3 grams/cm³, most preferably from 0.08 grams/cm³ to 0.25 grams/cm³; and/or
a Specific Surface Area of from 0.03 m²/g to 0.25 m²/g, preferably from 0.04 m²/g to 0.22 m²/g, more preferably from 0.05 m²/g to 0.2 m²/g, most preferably from 0.1 m²/g to 0.18 m²/g.

In some embodiments, the flexible, porous, dissolvable solid sheet article further comprises a loading composition in a form of paste or powders between adjacent two sheets.

In some embodiments, the loading composition is in a form of non-aqueous paste and comprises a non-aqueous liquid carrier and solid particles. Preferably, said non-aqueous paste comprises: from 1% to 99%, preferably from 5% to 70%, more preferably from 10% to 60%, of a non-aqueous liquid carrier by total weight of said non-aqueous paste; and/or from 1% to 99%, preferably from 10% to 80%, more preferably from 30% to 75%, of solid particles by total weight of said non-aqueous paste.

In some embodiments, said non-aqueous liquid carrier is selected from the group consisting of polyethylene glycol, polypropylene glycol, silicone, fatty acid, perfume oil, a non-ionic surfactant, an organic solvent and any combinations thereof, preferably wherein said non-aqueous liquid carrier comprises a non-ionic surfactant that is preferably selected from the group consisting of C₆-C₂₀ linear or branched alkylalkoxylated alcohols (AA) having a weight average degree of alkoxylation ranging from 5 to 15.

In some embodiments, said solid particles comprise an oxidative dye compound, a pH modifier and/or a buffering agent, a radical scavenger, a chelant, a warming active, a color indicator, an anionic surfactant, an enzyme, a bleaching agent, an effervescent system and any combinations thereof. Preferably, said solid particles comprises C₆-C₂₀ linear alkylbenzene sulphonate (LAS) surfactant, percarbonate salts, perborate salts, persulfate salts, tetraacetylethylenediamine (TAED), oxybenzene sulphonates, caprolactams, or any combinations thereof.

In some embodiments, the loading composition is in a form of powders which are characterized by a bulk density of from 250 g/l to 500 g/l, e.g., from 300 g/l to 500 g/l, from 350 g/l to 500 g/l. Preferably, the powders are characterized by a mean particle size of from about 200 to about 600 microns, preferably from about 300 to about 500 microns.

In some embodiments, said powders comprises an anionic surfactant which is preferably selected from the group consisting of C₆-C₂₀ linear alkylbenzene sulfonate (LAS), a C₆-C₂₀ linear or branched alkylalkoxy sulfates (AAS) having a weight average degree of alkoxylation ranging from 0.5 to 10, a C₆-C₂₀ linear or branched alkyl sulfates (AS) and any combinations thereof.

In some embodiments, said powders comprises an additional surfactant, a perfume, a chelant, a polymer, silica, an antioxidant, a builder, an enzyme, an inorganic acid, an oxidative dye compound, a radical scavenger, a color indicator, a bleaching agent, an effervescent system or any combinations thereof.

In some embodiments, the C₆-C₂₀ linear or branched alkylalkoxy sulfates (AAS) having a weight average degree of alkoxylation ranging from 0.5 to 10 is selected from the group consisting of linear or branched alkylethoxy sulfates (AES) having the respective formulae RO(C₂H₄O)ₓSO₃M, wherein R is alkyl or alkenyl of from about 6 to about 18, preferably from about 8 to about 16, more preferably from about 10 to about 14, carbon atoms, x is from 1 to 10, preferably from 1 to 7, more preferably from 1 to 5, and M is a water-soluble cation such as ammonium, sodium, potassium and triethanolamine.

In some embodiments, the C₆-C₂₀ linear or branched alkyl sulfates (AS) is selected from the group consisting of alkyl sulfates having the respective formulae ROSO₃M, wherein R is alkyl or alkenyl of from about 6 to about 18, preferably from about 8 to about 16, more preferably from about 10 to about 14, carbon atoms, and M is a water-soluble cation such as ammonium, sodium, potassium and triethanolamine.

It is advantageous that the flexible, porous, dissolvable solid sheet article according to the present disclosure can have a desirable porous solid structure.

It is further advantageous that the flexible, porous, dissolvable solid sheet article according to the present disclosure can be compatible with the continuous manufacturing process according to the present disclosure.

It is another advantage of the flexible, porous, dissolvable solid sheet article according to the present disclosure that it may achieve an improved performance in the aspect of delamination.

### BRIEF DESCRIPTION OF THE DRAWINGS

FIG. 1 is an exemplary embodiment of the equipment used for practicing a continuous process according to the present disclosure.

FIG. 2 shows multiple heating components in an exemplary system of belt drying according to the present disclosure.

FIG. 3 shows an exemplary system for making a multilayer unit dose article.

FIG. 4 shows an exemplary tooling used to form the edge seal of the article.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

### Definitions

The flexible porous dissolvable solid structure article may be referred to herein as “the Article” or “the Dissolvable Article”. All references are intended to mean the flexible dissolvable porous solid structure article.

The term “flexible” as used herein refers to the ability of an article to withstand stress without breakage or significant fracture when it is bent at 90° along a center line perpendicular to its longitudinal direction. Preferably, such article can undergo significant elastic deformation and is characterized by a Young's Modulus of no more than 5 GPa, preferably no more than 1 GPa, more preferably no more than 0.5 GPa, most preferably no more than 0.2 GPa.

The term “dissolvable” as used herein refers to the ability of an article to completely or substantially dissolve in a sufficient amount of deionized water at 20° C. and under the atmospheric pressure within eight (8) hours without any stirring, leaving less than 5 wt % undissolved residues.

The term “solid” as used herein refers to the ability of an article to substantially retain its shape (i.e., without any visible change in its shape) at 20° C. and under the atmospheric pressure, when it is not confined and when no external force is applied thereto.

The term “sheet” as used herein refers to a non-fibrous structure having a three-dimensional shape, i.e., with a thickness, a length, and a width, while the length-to-thickness aspect ratio and the width-to-thickness aspect ratio are both at least about 5:1, and the length-to-width ratio is at least about 1:1. Preferably, the length-to-thickness aspect ratio and the width-to-thickness aspect ratio are both at least about 10:1, more preferably at least about 15:1, most preferably at least about 20:1; and the length-to-width aspect ratio is preferably at least about 1.2:1, more preferably at least about 1.5:1, most preferably at least about 1.618:1.

As used herein, the term “continuous” process refers to a manufacturing method where the production of a product is ongoing without a defined start or endpoint. The term “batch” process refers to a manufacturing method where a specific quantity of goods are made in a single production run. It has a defined start and endpoint, meaning the process is completed once the batch has been produced.

As used herein, the term “bottom surface” refers to a surface of the flexible, porous, dissolvable solid sheet article of the present invention that is immediately contacting a supporting surface upon which the sheet of aerated wet pre-mixture is placed during the drying step, while the term “top surface” refers to a surface of the sheet article that is opposite to the bottom surface. Further, such solid sheet article can be divided into three (3) regions along its thickness, including a top region that is adjacent to its top surface, a bottom region that is adjacent to its bottom surface, and a middle region that is located between the top and bottom regions. The top, middle, and bottom regions are of equal thickness, i.e., each having a thickness that is about ⅓ of the total thickness of the sheet article.

The term “open celled foam” or “open cell pore structure” as used herein refers to a solid, interconnected, polymer-containing matrix that defines a network of spaces or cells that contain a gas, typically a gas (such as air), without collapse of the foam structure during the drying process, thereby maintaining the physical strength and cohesiveness of the solid. The interconnectivity of the structure may be described by a Percent Open Cell Content, which is measured by Test 3 disclosed hereinafter.

The term “water-soluble” as used herein refers to the ability of a sample material to completely dissolve in or disperse into water leaving no visible solids or forming no visibly separate phase, when at least about 25 grams, preferably at least about 50 grams, more preferably at least about 100 grams, most preferably at least about 200 grams, of such material is placed in one liter (1 L) of deionized water at 20° C. and under the atmospheric pressure with sufficient stirring.

The term “aerate”, “aerating” or “aeration” as used herein refers to a process of introducing a gas into a liquid or pasty composition by mechanical and/or chemical means.

The term “heating direction” as used herein refers to the direction along which a heat source applies thermal energy to an article, which results in a temperature gradient in such article that decreases from one side of such article to the other side. For example, if a heat source located at one side of the article applies thermal energy to the article to generate a temperature gradient that decreases from the one side to an opposing side, the heating direction is then deemed as extending from the one side to the opposing side. If both sides of such article, or different sections of such article, are heated simultaneously with no observable temperature gradient across such article, then the heating is carried out in a non-directional manner, and there is no heating direction.

The term “substantially opposite to” or “substantially offset from” as used herein refers to two directions or two lines having an offset angle of 90° or more therebetween.

The term “substantially aligned” or “substantial alignment” as used herein refers to two directions or two lines having an offset angle of less than 90° therebetween.

The term “age” or “aging” as used herein refers to a process of maintaining an aerated wet mixture or pre-mixture for a while without further introducing a significant amount of gas. Preferably, the aging may be conducted under the conditions of being essentially free of mechanical energy input and/or being essentially free of heat input. More preferably, the aging may be conducted under the ambient temperature without any stirring.

The term “essentially free of” or “essentially free from” means that the indicated material is at the very minimal not deliberately added to the composition or product, or preferably not present at an analytically detectible level in such composition or product. It may include compositions or products in which the indicated material is present only as an impurity of one or more of the materials deliberately added to such compositions or products.

The test methods disclosed in the Test Methods Section of the present application should be used to determine the respective values of the parameters of Applicants' inventions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Method of Manufacture

The water-soluble sheet according to the present disclosure can be made by using known methods. Particularly, a water-soluble film can be made by known method. Such water-soluble film preferably comprises polyvinyl alcohol or a copolymer thereof. Preferably, the water-soluble film comprises a blend of at least two different polyvinylalcohol homopolymers, at least two different polyvinylalcohol copolymers, at least one polyvinylalcohol homopolymer and at least one polyvinylalcohol copolymer or a combination thereof. Preferably, the water-soluble film has a thickness between 50 microns and 100 microns, preferably between 70 microns and 90 microns before being deformed into a unit dose article. WO2019/147529 discloses a method for making a multi-layer fibrous water-soluble products containing fibrous sheets. The fibrous sheet has a fibrous structure comprising one or more fibrous elements. The fibrous elements can be associated with one another to form a structure.

With respect to the porous sheets, WO2010077627 discloses a batch process for forming porous sheets with open-celled foam (OCF) structures. WO2012138820 discloses a similar process as that of WO2010077627, except that continuous drying of the aerated wet pre-mixture is achieved by using, e.g., an impingement oven (instead of a convection oven or a microwave oven). Furthermore, WO2021/102935 discloses another drying process for making the porous sheets. A typical method for making flexible, porous, dissolvable solid sheets may comprise the steps of: (a) forming a pre-mixture containing raw materials (e.g., the water-soluble polymer, active ingredients such as surfactants, and optionally a plasticizer) dissolved or dispersed in water or a suitable solvent, which is characterized by a viscosity of from about 1,000 cps to about 25,000 cps measured at about 40° C. and 1 s⁻¹; (b) aerating the pre-mixture (e.g., by introducing a gas into the wet slurry) to form an aerated wet pre-mixture; (c) forming the aerated wet pre-mixture into a sheet having opposing first and second sides; and (d) drying the formed sheet for a drying time of from 1 minute to 60 minutes at a temperature from 70° C. to 200° C. along a heating direction that forms a temperature gradient decreasing from the first side to the second side of the formed sheet, wherein the heating direction is substantially offset from the gravitational direction for more than half of the drying time, i.e., the drying step is conducted under heating along a mostly “anti-gravity” heating direction. Such a mostly “anti-gravity” heating direction can be achieved by various means, which include but are not limited to the bottom conduction-based heating/drying arrangement and the rotary drum-based heating/drying arrangement.

The Article can be prepared by the continuous process comprising: a) preparing a wet pre-mixture comprising a water-soluble polymer and a surfactant and having a viscosity of from 1,000 cps to 25,000 cps measured at 40° C. and 1 s⁻¹; b) acrating the wet pre-mixture to form an aerated wet pre-mixture having a 5 of from 0.05 to 0.5 g/ml; c) forming the aerated wet pre-mixture into a sheet having a top side and a bottom side, for example by extruding the aerated wet pre-mixture; and d) drying the formed sheet of aerated wet pre-mixture on a conveying belt with the bottom side of the formed sheet contacting the conveying belt.

### A. Preparation of Pre-Mixture

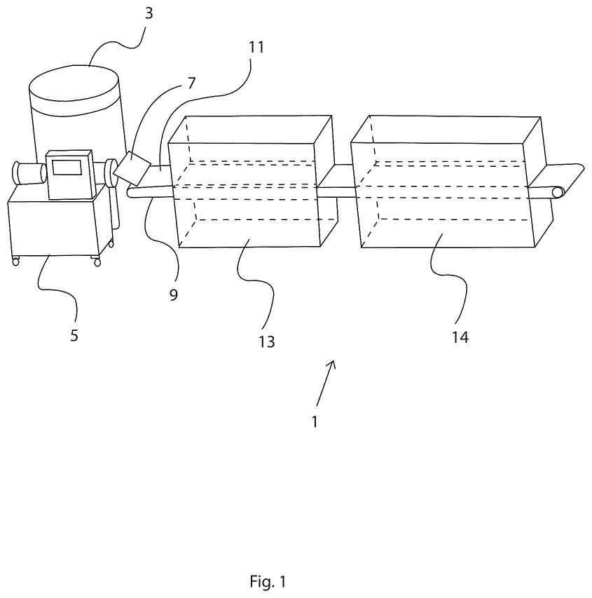

FIG. 1 depicts an exemplary embodiment of the equipment useful for practicing a continuous process for generating an Article. As shown in FIG. 1, in a continuous manufacturing equipment 1, the solids of interest are mixed in a premix tank 3. The pre-mixture is generally prepared by mixing the solids of interest, including surfactant(s), dissolved water soluble polymer, optional plasticizer and other optional ingredients.

In one embodiment, the pre-mixture can be formed using a mechanical mixer. Mechanical mixers useful herein, include, but aren't limited to pitched blade turbines or MAXBLEND mixer (Sumitomo Heavy Industries).

For addition of the ingredients in the pre-mixture, it can be envisioned that the polymer is ultimately dissolved in the presence of water, the surfactant(s), optional actives, optional plasticizer, and any other optional ingredients including step-wise processing via pre-mix portions of any combination of ingredients.

### B. Optional Continued Heating of Pre-Mixture

Optionally, the pre-mixture is pre-heated immediately prior to the aeration process at above ambient temperature but below any temperatures that would cause degradation of the component.

In one embodiment, the pre-mixture is kept at above about 40° C. and below about 99° C., in another embodiment above about 50° C. and below about 95° C., in another embodiment from about 60° C. and below about 90° C. In one embodiment, when the viscosity at ambient temperature of the pre-mix is from about 1000 cps to about 20,000 cps, the optional continuous heating is utilized before the aeration step. In an additional embodiment, additional heat is applied during the aeration process to try and maintain an elevated temperature during the aeration. This can be accomplished via conductive heating from one or more surfaces, injection of steam or other processing means.

### C. Aeration of Pre-Mixture

The aeration of the pre-mixture may be accomplished by introducing a gas into the pre-mixture in one embodiment by mechanical mixing energy but also may be achieved via chemical means to form an aerated mixture. As shown in FIG. 1, the aeration of the pre-mixture is achieved by an aeration unit 5. The aeration may be accomplished by any suitable mechanical processing means, including but not limited to: (i) batch tank aeration via mechanical mixing including planetary mixers or other suitable mixing vessels, or (ii) semi-continuous or continuous aerators utilized in the food industry (pressurized and non-pressurized), or (iii) spray-drying the processing mixture in order to form aerated beads or particles that can be compressed such as in a mould with heat in order to form the porous solid.

In another embodiment, aeration with chemical foaming agents by in-situ gas formation (via chemical reaction of one or more ingredients, including formation of carbon dioxide (CO₂ (g)) by an effervescent system) can be used.

In a particular embodiment, it has been discovered that the Article can be prepared within continuous pressurized aerators that are conventionally utilized in the foods industry in the production of marshmallows. Suitable continuous pressurized aerators include the Morton whisk (Morton Machine Co., Motherwell, Scotland), the Oakes continuous automatic mixer (E.T. Oakes Corporation, Hauppauge, New York), the Fedco Continuous Mixer (The Peerless Group, Sidney, Ohio), the Mondo (Haas-Mondomix B.V., Netherlands), the Acros (Acros Industrial Equipment Co., Ltd., Guangdong Province, China), and the Preswhip (Hosokawa Micron Group, Osaka, Japan). Continuous mixers may work to homogenize or aerate slurry to produce highly uniform and stable foam structures with uniform bubble sizes. The unique design of the high shear rotor/stator mixing head may lead to uniform bubble sizes in the layers of the open celled foam.

### D. Forming the Aerated Wet Pre-Mixture

As seen in FIG. 1, the forming of the aerated wet pre-mixture is accomplished by extruding the aerated mixture through an extrusion nozzle 7 onto a continuous belt 9 or screen comprising any non-interacting or non-stick material such as solid metallic materials, flexible plastic materials including infrared transparent materials, and combinations thereof. Nonlimiting examples of solid metallic materials include stainless steel. Nonlimiting examples of flexible plastic materials including but are not limited to materials such as HDPE, polycarbonate, NEOPRENE®, rubber, LDPE, and fiberglass. Nonlimiting examples of infrared transparent materials include but are not limited to TEFLON®.

After extrusion, the aerated wet pre-mixture forms one or more sheets. In one embodiment, one sheet 11 forms the Article having a thickness of from about 0.5 mm to about 20 mm. In another embodiment, the Article has a thickness of about 1 to 2 mm. In another embodiment, two or more sheets 11 are combined to form an Article having a final thickness from about 2 mm to about 10 mm. The extrusion of thinner sheets that are then combined to form an Article allows for a faster drying time for the individual sheets. The sheets can be combined by any means known in the art, examples of which include but are not limited to, chemical means, mechanical means, and combinations thereof. The combination of the sheets allows for two or more sheets to be stacked on top of one another.

The wet density range of the aerated pre-mixture ranges from about 0.05 g/cm³ to about 0.5 g/cm³, from about 0.10 g/cm³ to about 0.45 g/cm³, from about 0.20 g/cm³ to about 0.40 g/cm³, and from about 0.25 g/cm³ to about 0.35 g/cm³.

### E. Step-Wise Drying the Formed Aerated Wet Pre-Mixture

The drying of the formed aerated wet pre-mixture according to the present application is a step-wise process. Particularly, the conveying belt is configured to sequentially pass through multiple heating zones with heating temperatures ranging from 70° C. to 200° C.; wherein said multiple heating zones comprises a first heating zone and a second heating zone which is located downstream of said first heating zone. More particularly, said first heating zone is configured to simultaneously heat the top and bottom sides of said formed sheet at a first top heating temperature (Tₜ₁) and a first bottom heating temperature (T_{b1}) for a first heating duration of from 0.01 minutes to 20 minutes; wherein said second heating zone is configured to simultaneously heat the top and bottom sides of said formed sheet at a second top heating temperature (Tₜ₂) and a second bottom heating temperature (T_{b2}) for a second heating duration of from 0.01 minutes to 20 minutes; and wherein T_{b1}>Tₜ₁; T_{b1}>T_{b2}; and Tₜ₁<Tₜ₂.

In some embodiments, the first top heating temperature (Tₜ₁) ranges from 70° C. to 160° C.; the first bottom heating temperature (T_{b1}) ranges from 80° C. to 190° C.; the second top heating temperature (Tₜ₂) ranges from 100° C. to 200° C.; and the second bottom heating temperature (T_{b2}) ranges from 70° C. to 170° C.

In some embodiments, Tₜ₁ ranges from 80° C. to 150° C., preferably from 80° C. to 140° C., more preferably from 90° C. to 120° C., for example 80° C., 90° C., 100° C., 110° C., 120° C., 130° C., 140° C., 150° C. or any ranges therebetween; wherein T_{b1} ranges from 90° C. to 170° C., preferably from 100° C. to 160° C., more preferably from 110° C. to 140° C., for example 90° C., 100° C., 110° C., 120° C., 130° C., 140° C., 150° C., 160° C. or any ranges therebetween; wherein Tₜ₂ ranges from 110° C. to 190° C., preferably from 120° C. to 180° C., more preferably from 130° C. to 160° C., for example 110° C., 120° C., 130° C., 140° C., 150° C., 160° C., 170° C., 180° C., or any ranges therebetween; and wherein T_{b2} ranges from 70° C. to 150° C., preferably from 70° C. to 120° C., more preferably from 70° C. to 110° C., for example 70° C., 80° C., 90° C., 100° C., 110° C., 120° C., 130° C., 140° C., 150° C. or any ranges therebetween. Preferably, T_{b2}≤Tₜ₂.

In some embodiments, said multiple heating zones further comprises a third heating zone which is located downstream of said second heating zone and wherein said conveying belt is configured to pass through said third heating zone; wherein said third heating zone is configured to simultaneously heat the top and bottom sides of said formed sheet at a third top heating temperature (Tₜ₃) and a third bottom heating temperature (T_{b3}) for a third heating duration of from 0.01 minutes to 20 minutes; and wherein T_{b2}>T_{b3}; Tₜ₃>Tₜ₂; and T_{b3}<Tₜ₃.

In some embodiments, Tₜ₃ ranges from 120° C. to 200° C., preferably from 130° C. to 190° C.; and wherein T_{b3} ranges from 70° C. to 150° C., preferably from 70° C. to 120° C. In some embodiments, the multiple heating zones comprises 4, 5, 6, 7, 8, 9, 10, 15, 20, 25, 30 or more heating zones in total in which the n^{th} heating zone is configured to simultaneously heat the top and bottom sides of said formed sheet at a n^{th} top heating temperature (Tₜₙ) and a n^{th} bottom heating temperature (T_{bn}), and wherein T_{bn}≥T_{b(n+1)}; and Tₜₙ≤Tₜ₍ₙ₊₁₎. Preferably, the multiple heating zones comprises 0.1 to 5 (for example 0.2, 0.3, 0.5, 1, 1.5, 2, 2.5, 3, 3.5, 4, 4.5, 5 or any ranges therebetween) heating zones per meter of the conveying belt.

In some embodiments, said first heating duration is from 0.05 minutes to 10 minutes, preferably from 0.1 minutes to 8 minutes; and/or said second heating duration is from 0.05 minutes to 10 minutes, preferably from 0.1 minutes to 8 minutes; and/or said third heating duration from 0.05 minutes to 10 minutes, preferably from 0.1 minutes to 8 minutes; and/or the total heating duration in said multiple heating zones is from 0.05 minutes to 30 minutes, preferably from 0.1 minutes to 20 minutes, more preferably from 0.15 minutes to 15 minutes.

In some embodiments, said formed sheet of aerated wet pre-mixture is characterized by a thickness ranging from 0.5 mm to 20 mm, preferably from 0.8 mm to 15 mm, more preferably from 1 mm to 10 mm, still more preferably from 1.2 mm to 8 mm, most preferably from 1.4 mm to 6 mm, e.g. 1.5 mm, 2 mm, 2.5 mm, 3 mm, 3.5 mm, 4 mm, 5 mm or any ranges therebetween. In a preferred embodiment, the formed sheet is characterized by a thickness ranging from 1.5 mm to 20 mm, preferably from 1.5 mm to 15 mm, more preferably from 1.5 mm to 10 mm, still more preferably from 1.5 mm to 8 mm, most preferably from 1.5 mm to 6 mm.

In some embodiments, said wet pre-mixture comprises from 3% to 70%, preferably from 4% to 50%, more preferably from 5% to 40%, of said water-soluble polymer by total weight of said wet pre-mixture; and/or said wet pre-mixture comprises from 1% to 40%, preferably from 2% to 35%, more preferably from 5% to 30%, of said surfactant by total weight of said wet pre-mixture; and/or the density of the aerated wet pre-mixture is from 0.08 to 0.4 g/ml, preferably from 0.1 to 0.35 g/ml; and/or the wet pre-mixture is characterized by a solid content ranging from 15% to 70%, preferably from 20% to 50%, more preferably from 25% to 45% by weight of said wet pre-mixture; and/or the wet pre-mixture is characterized by a viscosity ranging from 3,000 cps to 24,000 cps, preferably from 5,000 cps to 23,000 cps, more preferably from 10,000 cps to 20,000 cps as measured at 40° C. and 1 s⁻¹.

As seen in FIG. 1, the drying of the formed aerated wet pre-mixture may be accomplished by any suitable drying environments 13 and 14 including, but not limited to (i) drying room(s) including rooms with controlled temperature and pressure or atmospheric conditions; (ii) ovens including non-convection or convection ovens with controlled temperature and optionally humidity; (iii) Truck/Tray driers, (iv) multi-stage inline driers; (v) impingement ovens; (vi) rotary ovens/driers; (vii) inline roasters; (viii) rapid high heat transfer ovens and driers; (ix) dual plenum roasters; (x) conveyor driers; (xi) vacuum drying chambers; (xii) air distribution plate; (xiii) venturi dryers and combinations thereof. The drying environments 13 and 14 can provide different heating temperatures.

In one embodiment, the drying environments 13 and 14 are selected from the group consisting of one or more drying rooms, convection ovens, multi-tier ovens, Truck/Tray driers, multi-stage inline driers, impingement ovens/driers, rotary ovens/driers, inline roasters, rapid high heat transfer ovens and driers, dual plenum roasters, conveyor driers, vacuum drying chambers and combinations thereof, such that the drying environment is between 100° C. and 150° C.

Other suitable drying environments include “volumetric heating” techniques using high frequency electromagnetic fields such as microwave drying and infrared drying. With these techniques, the energy is transferred electromagnetically through the aerated wet pre-mixture rather than by conduction or convection.

In some embodiments, the first top heating temperature (Tₜ₁) in the drying environment 13 ranges from 70° C. to 160° C.; the first bottom heating temperature (T_{b1}) in the drying environment 13 ranges from 80° C. to 190° C.; the second top heating temperature (Tₜ₂) in the drying environment 14 ranges from 100° C. to 200° C.; and the second bottom heating temperature (T_{b2}) in the drying environment 14 ranges from 70° C. to 170° C.

In some embodiments, Tu ranges from 80° C. to 150° C., preferably from 80° C. to 140° C.; wherein T_{b1} ranges from 90° C. to 170° C., preferably from 100° C. to 160° C.; wherein Tre ranges from 110° C. to 190° C., preferably from 120° C. to 180° C.; and wherein T_{b2} ranges from 70° C. to 150° C., preferably from 70° C. to 120° C.; and wherein T_{b2}≤Tₜ₂.

In some embodiments, said multiple heating zones further comprises a third heating zone and wherein said conveying belt is configured to pass through said third heating zone; wherein said third heating zone is configured to simultaneously heat the top and bottom sides of said formed sheet at a third top heating temperature (Tₜ₃) and a third bottom heating temperature (T_{b3}) for a third heating duration of from 0.01 minutes to 20 minutes.

In some embodiments, T_{b2}≥T_{b3}; Tₜ₂≤Tₜ₃; and T_{b3}≤Tₜ₃ when said third heating zone is located downstream of said second heating zone.

In some embodiments, T_{b1}≥T_{b3}≥T_{b2}; Tₜ₁≤Tₜ₃≤Tₜ₂ when said third heating zone is located downstream of said first heating zone and upstream of said second heating zone.

In some embodiments, T_{b3}≥T_{b1}; Tₜ₃≤Tₜ₁; and T_{b3}≥Tₜ₃ when said third heating zone is located upstream of said first heating zone.

In some embodiments, To ranges from 90° C. to 200° C.; and wherein T_{b3} ranges from 70° C. to 180° C.

In some embodiments, said first heating duration is from 0.1 minutes to 10 minutes, preferably from 0.15 minutes to 8 minutes; and/or said second heating duration is from 0.1 minutes to 10 minutes, preferably from 0.15 minutes to 8 minutes; and/or said third heating duration is from 0.1 minutes to 10 minutes, preferably from 0.15 minutes to 8 minutes; and/or the total heating duration in said multiple heating zones is from 0.3 minutes to 30 minutes, preferably from 0.5 minutes to 20 minutes, more preferably from 0.6 minutes to 15 minutes.

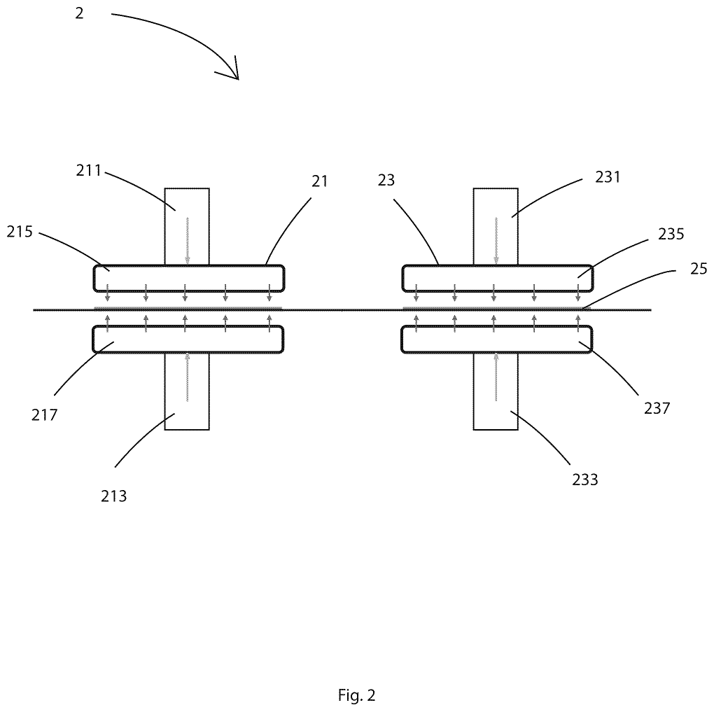

In an exemplary system of step-wise belt drying as shown in FIG. 2, the system 2 comprises a first heating zone 21 and a second heating zone 23. In the first heating zone 21, hot air is respectively introduced from a first top inlet 211 and a first bottom inlet 213 to a first top air distribution plate 215 and a first bottom air distribution plate 217 and then applied onto the top surface and the bottom surface of the belt as a hot air jet (as shown by the arrows). In the first heating zone 23, hot air is respectively introduced from a first top inlet 231 and a first bottom inlet 233 to a first top air distribution plate 235 and a first bottom air distribution plate 237 and then applied onto the top surface and the bottom surface of the belt as a hot air jet (as shown by the arrows). Particularly, the air distribution plates 215, 217, 235, and 237 respectively comprise a plurality of holes through which the hot air jet can be ejected onto the sheets formed on the belt or the belt itself for conductive and convective heating of the foam. Then, one dried solid sheet 25 is formed on the belt after drying.

The resulting Article also has an open celled foam with a percent open cells of from about 80% to about 100%. It has been unexpectedly found that the Article produced by the continuous process has uniformity in the upper, middle, and lower regions of the open celled foam.

The solid sheet article may comprise a top region adjacent to the top surface, a bottom region adjacent to the bottom surface, and a middle region therebetween; wherein the top, middle, and bottom regions have the same thickness, and each of the top, middle and bottom regions is characterized by an Average Pore Size (i.e., Top Average Pore Diameter, Middle Average Pore Diameter, and Bottom Average Pore Diameter). Particularly, the ratio of Average Pore Size in the bottom region over that in the top region may be from about 0.6 to about 1.5, preferably from about 0.7 to about 1.4, more preferably from about 0.8 to about 1.3, most preferably from about 0.9 to about 1.2; and/or the ratio of Average Pore Size in the bottom region over that in the middle region may be from about 0.6 to about 1.5, preferably from about 0.7 to about 1.4, more preferably from about 0.8 to about 1.3, most preferably from about 0.9 to about 1.2; and/or the ratio of Average Pore Size in the middle region over that in the top region may be from about 0.6 to about 1.5, preferably from about 0.7 to about 1.4, more preferably from about 0.8 to about 1.3, most preferably from about 0.9 to about 1.2.

In some embodiments, the solid sheet article is characterized by the Overall Average Pore Size is from 20 to 600 μm, preferably from 50 to 500 μm, more preferably from 100 to 400 μm, e.g. 100 μm, 120 μm, 150 μm, 180 μm, 200 μm, 250 μm, 300 μm, 350 μm, 400 μm or any ranges therebetween.

In some embodiments, the solid sheet article is characterized by the Standard Deviation of Overall Average Pore Diameter is from 20 to 250 μm, preferably from 30 to 250 μm, more preferably from 50 to 200 μm, e.g. 50 μm, 60 μm, 70 μm, 80 μm, 90 μm, 100 μm, 120 μm, 150 μm, 180 μm or any ranges therebetween.

F. Conversion of Water-Soluble Sheet and Loading Composition into Dissolvable Unit Dose Articles Containing Loading Composition

Once the water-soluble sheet of the present invention is formed, as described hereinabove, such sheet can be treated by loading a loading composition to form dissolvable unit dose articles of any desirable three-dimensional shapes, including but not limited to: spherical, cubic, rectangular, oblong, cylindrical, rod, sheet, flower-shaped, fan-shaped, star-shaped, disc-shaped, and the like. The sheets can be treated by any means known in the art, examples of which include but are not limited to, chemical means, mechanical means, and combinations thereof. Such treatment steps are hereby collectively referred to as a “conversion” process, i.e., which functions to convert such water-soluble sheet of the present invention into a dissolvable unit dose article containing a loading composition.

The loading composition can be in a form of paste comprising a non-aqueous liquid carrier, solid particles and a polyalkylene polymer, or in a form of powders comprising solid particles.

The loading composition may comprise from 1% to 99%, preferably from 5% to 70%, more preferably from 20% to 50%, for example 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or any ranges therebetween, of the non-aqueous liquid carrier by total weight of the loading composition. The non-aqueous liquid carrier may be selected from the group consisting of polyethylene glycol, polypropylene glycol, silicone, fatty acid, perfume oil, a non-ionic surfactant, an organic solvent and any combinations thereof.

The above-described multilayer dissolvable unit dose article may comprise more than two of sheets. For example, it may comprise from about 3 to about 50, preferably from about 4 to about 40, more preferably from about 5 to about 30, for example 6, 7, 8, 9, 10, 15, 20, 25, 30 or any ranges therebetween, of the sheets.

In some preferred embodiments, the multiple layers of the multilayer dissolvable unit dose article may be bonded together through heat-compressing when a loading composition is contained between layers. In some other embodiments, the multilayer dissolvable unit dose article may further comprise water-soluble thread at the edges which may help to seal the multiple layers together.

In a preferred embodiment, the dissolvable unit dose article comprises 2, 3, 4, 5, 6, 7, 8, 9, 10 or more water-soluble sheets in which one or more pastes are loaded between adjacent sheets. For example, the dissolvable unit dose article comprises 5 sheets in which two pastes are respectively loaded between 2^{nd} and 3^{rd} sheets and between 3^{rd} and 4^{th} sheets.

In another embodiment, the dissolvable unit dose article may further comprise a loading composition in a form of powders. Preferably, the loading composition in a form of powders is characterized by a bulk density of from 250 g/l to 500 g/l, e.g., from 300 g/l to 500 g/1, from 350 g/l to 500 g/l.

Particularly, the loading composition may be applied between individual sheets of the multilayer dissolvable solid article by any appropriate means, e.g., by spraying, sprinkling, dusting, coating, spreading, dipping, injecting, rolling, or even vapor deposition. More particularly, the loading composition may be applied on one or both of contacting surfaces of adjacent sheets in the stack. In a preferred embodiment, in order to avoid interference of the loading composition with the cutting seal or edge seal near the peripherals of the individual sheets, the loading composition may be applied in a central region on each of the applied surfaces of the respective sheets, which is preferably defined as a region that is spaced apart from the peripherals of such adjacent sheets by a distance that is at least 5%, preferably at least 10%, more preferably at least 15%, most preferably at least 20%, of the maximum Dimension D. In an alternative preferred embodiment, said loading composition is applied throughout the applied surfaces of the respective sheets, preferably wherein the applied area accounts for at least 90%, preferably 95%, more preferably 98%, most preferably 99% of the total area of the applied surfaces.

In a preferred embodiment, the loading composition may be applied on one or both contacting surfaces of any adjacent sheets in the solid article. In another preferred embodiment, the loading composition may be applied on one or both contacting surfaces of middle two sheets in the stack. In yet another preferred embodiment, the loading composition may be applied on one or both of contacting surfaces of any two adjacent sheets in the stack excluding the two outermost sheets.

In some preferred embodiments, the sheet in the unit dose article according to the present disclosure is relatively high compressible. Without wishing to be bound by theory, it is believed that the sheet being relatively high compressible would result in an improved leakage performance compared to the sheet being relatively low compressible.

The conversion process according to the present disclosure may be achieved by a system for preparing dissolvable unit dose articles according to the present disclosure. A typical system for preparing dissolvable unit dose articles according to the present disclosure may comprise a belt conveyor, a loading unit, an edge sealing unit, and optionally, an embossing unit. Particularly, the loading unit may comprise a nozzle and a flattening mechanism in which the nozzle is configured to load a loading composition in a form of paste onto a sheet and the flattening mechanism is configured to spread the loading composition evenly on the sheet. The loading unit may comprise a dispenser and a spreading roller in which the dispenser is configured to load a loading composition in a form of powders onto a sheet and the spreading roller is configured to spread the loading composition evenly on the sheet.

In some embodiments, the system may further comprise one or more compression rollers which are configured to compress the stack of the sheets. Particularly, the compression rollers can be located at anywhere after two sheets are fed on the belt conveyor.

In some embodiments, the system may further comprise one or more spraying units which are configured to spray an aqueous liquid (e.g. water) onto sheets. The addition of the aqueous liquid may be helpful in combining multiple sheets into a dissolvable unit dose article and improve the leakage of loading compositions.

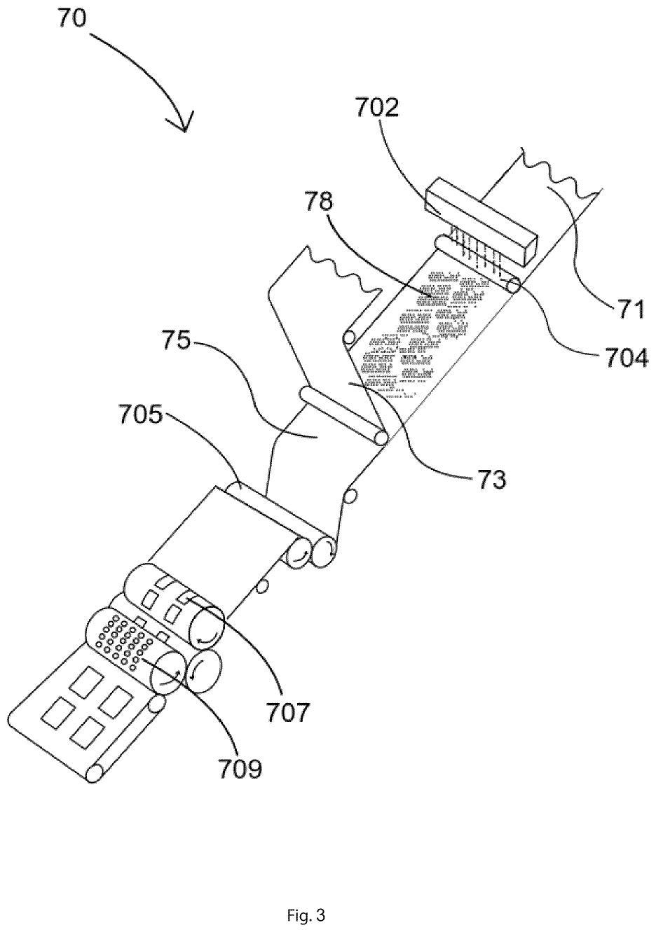

FIG. 3 shows an exemplary system for preparing multilayer dissolvable unit dose articles, wherein the system comprises a belt conveyor, a loading unit, an embossing unit which is configured to apply heat and pressure on the surface of sheets to achieve both bonding and embossing, and a cutting unit. Particularly, a first flexible, dissolvable, porous sheet 71, and a second flexible, dissolvable, porous sheet 73 are respectively fed on the belt conveyor sequentially. The loading unit comprises a dispenser 702 through which a loading composition in a form of powders may be loaded between the two sheets as well as a spreading roller 704 by which a loading composition 78 in the form of powder is spread evenly on the second sheet 73. Optionally, a stack 75 of sheets containing the loading composition therebetween pass through the embossing unit comprising an embossing roller 705 which can apply heat and pressure onto the stack 75 of sheets to make a pattern on the surface of the stack 75 of sheets. Finally, the stack of sheets passes through the edge sealing unit comprising an edge sealing roller 707 and a transferring roller 709. The edge sealing roller 707 comprises a tooling having a specific shape which can apply heat and pressure onto the stack of sheets so that the stack of sheets can be cut into unit dose articles with sealed edge. The transferring roller 709 is configured to transfer the unit dose articles to a next station through vacuum applied onto the transferring roller 709.

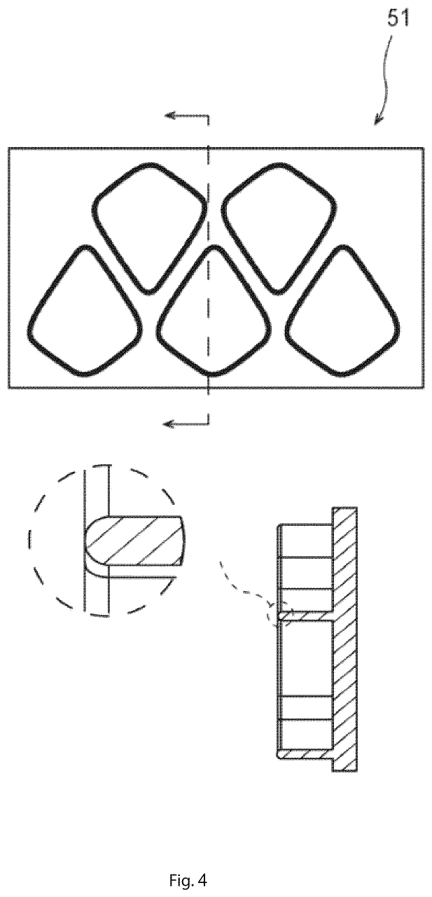

FIG. 4 shows an exemplary tooling 51 used to form the edge seal of the article which can be made of stainless steel. The top panel shows a top view of tooling 51 used to produce five unit dose articles. The bottom panel shows a cross-sectional view of the tooling bounded by the in the top panel as well as a cross-sectional detail of a tip of the tooling that comes in contact with the sheets to be sealed. Preferably, this tooling 51 may be bolted to a heated mechanical press which can apply both heating and pressure onto the sheets to be sealed. The heated mechanical press can be configured to apply a temperature of 50° C., 60° C., 70° C., 80° C., 90° C., 100° C., 110° C., 120° C., 130° C., 140° C., 150° C., 160° C., 170° C., 180° C., 190° C. or any ranges therebetween, and a pressure of 100 psi, 500 psi, 1000 psi, 3000 psi, 5000 psi, 7000 psi, 9000 psi, 10000 psi, 15000 psi, 20000 psi or any ranges therebetween. The duration of the contacting between the tooling 51 and the sheets can be set as 0.05, 0.1, 0.2, 0.3, 0.4, 0.5, 0.8, 1, 2, 3, 4, 5, 6, 7, 8, 9, 10 seconds or any ranges therebetween. The edge sealing can be one-step or two-step approach. In the two-step approach, the heated mechanical press can apply a first temperature and a first pressure in a first step to allow the edge sealing and then a second temperature and a second pressure in a second step to cut the unit dose articles out of the rest of the sheets. In some embodiments, the edge seal breadth can be half of the width of the tooling tip. For example, the width of the tip is 0.3 cm, and the edge seal breadth would be 0.15 cm. In some particular embodiments, the edge seal breadth can be 0.05 cm, 0.1 cm, 0.15 cm, 0.2 cm, 0.25 cm, 0.3 cm, 0.35 cm, 0.4 cm, 0.45 cm, 0.5 cm and any ranges therebetween.

In some other embodiments, the system for preparing dissolvable unit dose articles may further comprise a sewing unit which is configured to sew edges of dissolvable unit dose articles with water-soluble thread, e.g. water-soluble PVA thread.

### G. Further Optional Steps

Further optional steps not recited above may be added at any point during or after the recited process. Optional ingredients may be imparted during any of the above described four processing steps or even after the drying process.

Additional steps that can be used in the present process include cutting the resulting Article into smaller sizes, puncturing the Article with needles or slitting the Article. The size of the Article will depend upon the desired dosage amount of actives, or in this case surfactant. The frequency of perforation or slitting is confined to maintain the structural integrity of the Article such that it can still be handled.

The Article may be further manipulated into a shape or form other than a flat plane or sheet. Other three-dimensional shapes may include spherical bead or ball, flowers, flower petals, berry shapes and various known pasta shapes. As such the process may further include a step whereby the Article is manipulated into a three-dimensional shape.

The Article may be packaged for consumption individually or in a plurality of Articles. The Article may be included in a kit wherein various types of products are supplied, including Articles with different compositions, Article(s) with other products making up a regime of series of products for a desired benefit, or Article(s) with other products unrelated such as a toiletry travel kit for travel on airplanes.

Suitable packaging material may be selected such that the Article is protected from inadvertent exposure to liquids. The packaging material may be air and/or vapor permeable, dependent upon the environment in which the Article is to be sold.

The process may further include a step of packaging the Article individually for sale as a product. The process may further include a step of packaging a plurality of Article for sale as a product. The process may further include a step of including a packaged Article in a kit for sale as a product. The packaging step is undertaken after the formation of the Article, in one embodiment after the Article is cut into a suitable size. The Article may be packaged on the same line as the production of Article or the Article may be collected, shipped or stored, and then packaged at a later time.

### Composition of Article

### 1. Water-Soluble Polymer

The flexible, porous, dissolvable solid sheet of the present invention may be formed by a wet pre-mixture that comprises a water-soluble polymer and a first surfactant. Such a water-soluble polymer may function in the resulting solid sheet as a film-former, a structurant as well as a carrier for other active ingredients (e.g., surfactants, emulsifiers, builders, chelants, perfumes, colorants, and the like).

Preferably, the wet pre-mixture may comprise from about 3% to about 20% by weight of the pre-mixture of water-soluble polymer, in one embodiment from about 5% to about 15% by weight of the pre-mixture of water-soluble polymer, in one embodiment from about 7% to about 10% by weight of the pre-mixture of water-soluble polymer.

After drying, it is preferred that the water-soluble polymer is present in the flexible, porous, dissolvable solid sheet of the present invention in an amount ranging from about 5% to about 60%, preferably from about 7% to about 50%, more preferably from about 9% to about 40%, most preferably from about 10% to about 30%, for example 10%, 12%, 15%, 18%, 20%, 25%, 30% or any ranges therebetween, by total weight of the solid sheet. In a particularly preferred embodiment of the present invention, the total amount of water-soluble polymer(s) present in the flexible, porous, dissolvable solid sheet of the present invention is no more than 25% by total weight of such sheet.

Water-soluble polymers suitable for the practice of the present invention may be selected those with weight average molecular weights ranging from about 5,000 to about 400,000 Daltons, preferably from about 10,000 to about 300,000 Daltons, more preferably from about 15,000 to about 200,000 Daltons, most preferably from about 20,000 to about 150,000 Daltons. The weight average molecular weight is computed by summing the average molecular weights of each polymer raw material multiplied by their respective relative weight percentages by weight of the total weight of polymers present within the porous solid sheet. The weight average molecular weight of the water-soluble polymer used herein may impact the viscosity of the wet pre-mixture, which may in turn influence the bubble number and size during the aeration step as well as the pore expansion/opening results during the drying step. Further, the weight average molecular weight of the water-soluble polymer may affect the overall film-forming properties of the wet pre-mixture and its compatibility/incompatibility with certain surfactants.

The water-soluble polymers of the present invention may include, but are not limited to, synthetic polymers including polyvinyl alcohols, polyvinylpyrrolidones, polyalkylene oxides, polyacrylates, caprolactams, polymethacrylates, polymethylmethacrylates, polyacrylamides, polymethylacrylamides, polydimethylacrylamides, polyethylene glycol monomethacrylates, copolymers of acrylic acid and methyl acrylate, polyurethanes, polycarboxylic acids, polyvinyl acetates, polyesters, polyamides, polyamines, polyethyleneimines, maleic/(acrylate or methacrylate) copolymers, copolymers of methylvinyl ether and of maleic anhydride, copolymers of vinyl acetate and crotonic acid, copolymers of vinylpyrrolidone and of vinyl acetate, copolymers of vinylpyrrolidone and of caprolactam, vinyl pyrollidone/vinyl acetate copolymers, copolymers of anionic, cationic and amphoteric monomers, and combinations thereof.

The water-soluble polymers of the present invention may also be selected from naturally sourced polymers including those of plant origin examples of which include karaya gum, tragacanth gum, gum Arabic, acemannan, konjac mannan, acacia gum, gum ghatti, whey protein isolate, and soy protein isolate; seed extracts including guar gum, locust bean gum, quince seed, and psyllium seed; seaweed extracts such as Carrageenan, alginates, and agar; fruit extracts (pectins); those of microbial origin including xanthan gum, gellan gum, pullulan, hyaluronic acid, chondroitin sulfate, and dextran; and those of animal origin including casein, gelatin, keratin, keratin hydrolysates, sulfonic keratins, albumin, collagen, glutelin, glucagons, gluten, zein, and shellac.

Modified natural polymers can also be used as water-soluble polymers in the present invention. Suitable modified natural polymers include, but are not limited to, cellulose derivatives such as hydroxypropylmethylcellulose, hydroxymethylcellulose, hydroxyethylcellulose, methylcellulose, hydroxypropylcellulose, ethylcellulose, carboxymethylcellulose, cellulose acetate phthalate, nitrocellulose and other cellulose ethers/esters; and guar derivatives such as hydroxypropyl guar.

Preferred water-soluble polymers of the present invention include polyvinyl alcohols, polyvinylpyrrolidones, polyalkylene oxides, starch and starch derivatives, pullulan, gelatin, hydroxypropylmethylcelluloses, methycelluloses, and carboxymethycelluloses. More preferred water-soluble polymers of the present invention include polyvinyl alcohols, and hydroxypropylmethylcelluloses.

Most preferred water-soluble polymers of the present invention are polyvinyl alcohols characterized by a degree of hydrolysis ranging from about 40% to about 100%, preferably from about 50% to about 95%, more preferably from about 65% to about 92%, most preferably from about 70% to about 90%. Commercially available polyvinyl alcohols include those from Celanese Corporation (Texas, USA) under the CELVOL trade name including, but not limited to, CELVOL 523, CELVOL 530, CELVOL 540, CELVOL 518, CELVOL 513, CELVOL 508, CELVOL 504; those from Kuraray Europe GmbH (Frankfurt, Germany) under the Mowiol® and POVAL™ trade names; and PVA 1788 (also referred to as PVA BP17) commercially available from various suppliers including Lubon Vinylon Co. (Nanjing, China); and combinations thereof. In a particularly preferred embodiment of the present invention, the flexible, porous, dissolvable solid sheet comprises from about 10% to about 25%, more preferably from about 15% to about 23%, by total weight of such sheet, of a polyvinyl alcohol having a weight average molecular weight ranging from 80,000 to about 150,000 Daltons and a degree of hydrolysis ranging from about 80% to about 90%.

### 2. Surfactants

In addition to the water-soluble polymer described hereinabove, the solid sheet of the present invention comprises a surfactant. The surfactant may function as emulsifying agents during the aeration process to create a sufficient amount of stable bubbles for forming the desired OCF structure of the present invention. Further, the surfactant may function as active ingredients for delivering a desired cleansing benefit.

In a preferred embodiment of the present invention, the solid sheet comprises a surfactant selected from the group consisting of anionic surfactants, nonionic surfactants, cationic surfactants, zwitterionic surfactants, amphoteric surfactants, polymeric surfactants and any combinations thereof. Depending on the desired application of such solid sheet and the desired consumer benefit to be achieved, different surfactants can be selected. One benefit of the present invention is that the OCF structures of the solid sheet allow for incorporation of a high surfactant content while still providing fast dissolution. Consequently, highly concentrated cleansing compositions can be formulated into the solid sheets of the present invention to provide a new and superior cleansing experience to the consumers.

The surfactant as used herein may include both surfactants from the conventional sense (i.e., those providing a consumer-noticeable lathering effect) and emulsifiers (i.e., those that do not provide any lathering performance but are intended primarily as a process aid in making a stable foam structure). Examples of emulsifiers for use as a surfactant component herein include mono- and di-glycerides, fatty alcohols, polyglycerol esters, propylene glycol esters, sorbitan esters and other emulsifiers known or otherwise commonly used to stabilize air interfaces.

The total amount of the surfactant present in the solid sheet of the present invention may range widely from about 5% to about 95%, preferably from about 30% to about 90%, preferably from about 40% to about 80%, more preferably from about 50% to about 70%, e.g. 20%, 30%, 40%, 50%, 60%, 70%, 80% or any ranges therebetween, by total weight of the solid sheet. Correspondingly, the wet pre-mixture may comprise from about 1% to about 50% by weight of the wet pre-mixture of surfactant(s), in one embodiment from about 2% to about 40% by weight of the wet pre-mixture of surfactant(s), in one embodiment from about 10% to about 35% by weight of the wet pre-mixture of surfactant(s), in one embodiment from about 15% to about 30% by weight of the wet pre-mixture of surfactant(s).

Non-limiting examples of anionic surfactants suitable for use herein include alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, sodium lauroyl glutamate, and combinations thereof.

One category of anionic surfactants particularly suitable for practice of the present invention include C₆-C₂₀ linear alkylbenzene sulphonate (LAS) surfactant. LAS surfactants are well known in the art and can be readily obtained by sulfonating commercially available linear alkylbenzenes. Exemplary C₁₀-C₂₀ linear alkylbenzene sulfonates that can be used in the present invention include alkali metal, alkaline earth metal or ammonium salts of C₁₀-C₂₀ linear alkylbenzene sulfonic acids, and preferably the sodium, potassium, magnesium and/or ammonium salts of C₁₁-C₁₈ or C₁-C₁₄ linear alkylbenzene sulfonic acids. More preferred are the sodium or potassium salts of C₁₂ and/or C₁₄ linear alkylbenzene sulfonic acids, and most preferred is the sodium salt of C₁₂ and/or C₁₄ linear alkylbenzene sulfonic acid, i.e., sodium dodecylbenzene sulfonate or sodium tetradecylbenzene sulfonate.

LAS provides superior cleaning benefit and is especially suitable for use in laundry detergent applications. It has been a surprising and unexpected discovery of the present invention that when polyvinyl alcohol having a higher weight average molecular weight (e.g., from about 50,000 to about 400,000 Daltons, preferably from about 60,000 to about 300,000 Daltons, more preferably from about 70,000 to about 200,000 Daltons, most preferably from about 80,000 to about 150,000 Daltons) is used as the film-former and carrier, LAS can be used as a major surfactant, i.e., present in an amount that is more than 50% by weight of the total surfactant content in the solid sheet, without adversely affecting the film-forming performance and stability of the overall composition. Correspondingly, in a particular embodiment of the present invention, LAS is used as the major surfactant in the solid sheet. If present, the amount of LAS in the solid sheet of the present invention may range from about 10% to about 70%, preferably from about 20% to about 65%, more preferably from about 40% to about 60%, by total weight of the solid sheet.

Another category of anionic surfactants suitable for practice of the present invention include sodium trideceth sulfates (STS) having a weight average degree of alkoxylation ranging from about 0.5 to about 5, preferably from about 0.8 to about 4, more preferably from about 1 to about 3, most preferably from about 1.5 to about 2.5. Trideceth is a 13-carbon branched alkoxylated hydrocarbon comprising, in one embodiment, an average of at least 1 methyl branch per molecule. STS used by the present invention may be include ST(EOxPOy)S, while EOx refers to repeating ethylene oxide units with a repeating number x ranging from 0 to 5, preferably from 1 to 4, more preferably from 1 to 3, and while POy refers to repeating propylene oxide units with a repeating number y ranging from 0 to 5, preferably from 0 to 4, more preferably from 0 to 2. It is understood that a material such as ST2S with a weight average degree of ethoxylation of about 2, for example, may comprise a significant amount of molecules which have no ethoxylate, 1 mole ethoxylate, 3 mole ethoxylate, and so on, while the distribution of ethoxylation can be broad, narrow or truncated, which still results in an overall weight average degree of ethoxylation of about 2. STS is particularly suitable for personal cleansing applications, and it has been a surprising and unexpected discovery of the present invention that when polyvinyl alcohol having a higher weight average molecular weight (e.g., from about 50,000 to about 400,000 Daltons, preferably from about 60,000 to about 300,000 Daltons, more preferably from about 70,000 to about 200,000 Daltons, most preferably from about 80,000 to about 150,000 Daltons) is used as the film-former and carrier, STS can be used as a major surfactant, i.e., present in an amount that is more than 50% by weight of the total surfactant content in the solid sheet, without adversely affecting the film-forming performance and stability of the overall composition. Correspondingly, in a particular embodiment of the present invention, STS is used as the major surfactant in the solid sheet. If present, the amount of STS in the solid sheet of the present invention may range from about 10% to about 70%, preferably from about 20% to about 65%, more preferably from about 40% to about 60%, by total weight of the solid sheet.

Another category of anionic surfactants suitable for practice of the present invention include alkyl sulfates. These materials have the respective formulae ROSO₃M, wherein R is alkyl or alkenyl of from about 6 to about 20 carbon atoms, x is 1 to 10, and M is a water-soluble cation such as ammonium, sodium, potassium and triethanolamine. Preferably, R has from about 6 to about 18, preferably from about 8 to about 16, more preferably from about 10 to about 14, carbon atoms. Previously, unalkoxylated C₆-C₂₀ linear or branched alkyl sulfates (AS) have been considered the preferred surfactants in dissolvable solid sheets, especially as the major surfactant therein, due to its compatibility with low molecular weight polyvinyl alcohols (e.g., those with a weight average molecular weight of no more than 50,000 Daltons) in film-forming performance and storage stability. However, it has been a surprising and unexpected discovery of the present invention that when polyvinyl alcohol having a higher weight average molecular weight (e.g., from about 50,000 to about 400,000 Daltons, preferably from about 60,000 to about 300,000 Daltons, more preferably from about 70,000 to about 200,000 Daltons, most preferably from about 80,000 to about 150,000 Daltons) is used as the film-former and carrier, other surfactants, such as LAS and/or STS, can be used as the major surfactant in the solid sheet, without adversely affecting the film-forming performance and stability of the overall composition. Therefore, in a particularly preferred embodiment of the present invention, it is desirable to provide a solid sheet with no more than about 20%, preferably from 0% to about 10%, more preferably from 0% to about 5%, most preferably from 0% to about 1%, by weight of the solid sheet, of AS.

Another category of anionic surfactants suitable for practice of the present invention include C₆-C₂₀ linear or branched alkylalkoxy sulfates (AAS). Among this category, linear or branched alkyl ethoxy sulfates (AES) having the respective formulae RO(C₂H₄O)ₓSO₃M are particularly preferred, wherein R is alkyl or alkenyl of from about 6 to about 20 carbon atoms, x is 1 to 10, and M is a water-soluble cation such as ammonium, sodium, potassium and triethanolamine. Preferably, R has from about 6 to about 18, preferably from about 8 to about 16, more preferably from about 10 to about 14, carbon atoms. Particularly, x is 2 to 8, preferably from 3 to 7.

Nonionic surfactants that can be included into the solid sheet of the present invention may be any conventional nonionic surfactants, including but not limited to: alkyl alkoxylated alcohols, alkyl alkoxylated phenols, alkyl polysaccharides (especially alkyl glucosides and alkyl polyglucosides), polyhydroxy fatty acid amides, alkoxylated fatty acid esters, sucrose esters, sorbitan esters and alkoxylated derivatives of sorbitan esters, amine oxides, and the like. Preferred nonionic surfactants are those of the formula R¹(OC₂H₄)ₙOH, wherein R¹ is a C₈-C₁₈ alkyl group or alkyl phenyl group, and n is from about 1 to about 80. Particularly preferred are C₈-C₁₈ alkyl ethoxylated alcohols having a weight average degree of ethoxylation from about 1 to about 20, preferably from about 5 to about 15, more preferably from about 7 to about 10, such as NEODOL® nonionic surfactants commercially available from Shell. Other non-limiting examples of nonionic surfactants useful herein include: C₆-C₁₂ alkyl phenol alkoxylates where the alkoxylate units may be ethyleneoxy units, propyleneoxy units, or a mixture thereof; C₁₂-Cis alcohol and C₆-C₁₂ alkyl phenol condensates with ethylene oxide/propylene oxide block polymers such as Pluronic® from BASF; C₁₄-C₂₂ mid-chain branched alcohols (BA); C₁₄-C₂₂ mid-chain branched alkyl alkoxylates, BAEx, wherein x is from 1 to 30; alkyl polysaccharides, specifically alkyl polyglycosides; Polyhydroxy fatty acid amides; and ether capped poly(oxyalkylated) alcohol surfactants. Suitable nonionic surfactants also include those sold under the tradename Lutensol® from BASF.

The most preferred nonionic surfactants for practice of the present invention include C₆-C₂₀ linear or branched alkylalkoxylated alcohols (AA) having a weight average degree of alkoxylation ranging from 5 to 15, more preferably C₁₂-C₁₄ linear ethoxylated alcohols having a weight average degree of alkoxylation ranging from 7 to 9. If present, the amount of AA-type nonionic surfactant(s) in the solid sheet of the present invention may range from about 2% to about 40%, preferably from about 5% to about 30%, more preferably from about 8% to about 12%, by total weight of the solid sheet.

Amphoteric surfactants suitable for use in the solid sheet of the present invention includes those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate, and N-higher alkyl aspartic acids.

Zwitterionic surfactants suitable include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula:

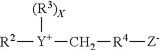

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Cationic surfactants can also be utilized in the present invention, especially in fabric softener and hair conditioner products. When used in making products that contain cationic surfactants as the major surfactants, it is preferred that such cationic surfactants are present in an amount ranging from about 2% to about 30%, preferably from about 3% to about 20%, more preferably from about 5% to about 15% by total weight of the solid sheet.

Cationic surfactants may include DEQA compounds, which encompass a description of diamido actives as well as actives with mixed amido and ester linkages. Preferred DEQA compounds are typically made by reacting alkanolamines such as MDEA (methyldiethanolamine) and TEA (triethanolamine) with fatty acids. Some materials that typically result from such reactions include N,N-di(acyl-oxyethyl)-N,N-dimethylammonium chloride or N,N-di(acyl-oxyethyl)-N,N-methylhydroxyethylammonium methylsulfate wherein the acyl group is derived from animal fats, unsaturated, and polyunsaturated, fatty acids.

Suitable polymeric surfactants for use in the personal care compositions of the present invention include, but are not limited to, block copolymers of ethylene oxide and fatty alkyl residues, block copolymers of ethylene oxide and propylene oxide, hydrophobically modified polyacrylates, hydrophobically modified celluloses, silicone polyethers, silicone copolyol esters, diquaternary polydimethylsiloxanes, and co-modified amino/polyether silicones.

In a preferred embodiment, the surfactant may be selected from the group consisting of a C₆-C₂₀ linear alkylbenzene sulfonate (LAS), a C₆-C₂₀ linear or branched alkylalkoxy sulfates (AAS) having a weight average degree of alkoxylation ranging from 0.5 to 10, a C₆-C₂₀ linear or branched alkylalkoxylated alcohols (AA) having a weight average degree of alkoxylation ranging from 5 to 15, a C₆-C₂₀ linear or branched alkyl sulfates (AS) and any combinations thereof.

### 3. Plasticizers

In a preferred embodiment of the present invention, the flexible, porous, dissolvable solid sheet of the present invention may further comprise a plasticizer, preferably in the amount ranging from about 0.1% to about 25%, preferably from about 0.5% to about 20%, more preferably from about 1% to about 15%, most preferably from 2% to 12%, by total weight of the solid sheet. Correspondingly, the wet pre-mixture used for forming such solid sheet may comprise from about 0.02% to about 20% of a plasticizer by weight of the wet pre-mixture, in one embodiment from about 0.1% to about 10% of a plasticizer by weight of the wet pre-mixture, in one embodiment from about 0.5% to about 5% of a plasticizer by weight of the wet pre-mixture.

Suitable plasticizers for use in the present invention include, for example, polyols, copolyols, polycarboxylic acids, polyesters, dimethicone copolyols, and the like.

Examples of useful polyols include, but are not limited to: glycerin, diglycerin, ethylene glycol, polyethylene glycol (especially 200-600), propylene glycol, butylene glycol, pentylene glycol, glycerol derivatives (such as propoxylated glycerol), glycidol, cyclohexane dimethanol, hexanediol, 2,2,4-trimethylpentane-1,3-diol, pentaerythritol, urea, sugar alcohols (such as sorbitol, mannitol, lactitol, xylitol, maltitol, and other mono- and polyhydric alcohols), mono-, di- and oligo-saccharides (such as fructose, glucose, sucrose, maltose, lactose, high fructose corn syrup solids, and dextrins), ascorbic acid, sorbates, ethylene bisformamide, amino acids, and the like.

Examples of polycarboxylic acids include, but are not limited to citric acid, maleic acid, succinic acid, polyacrylic acid, and polymaleic acid.

Examples of suitable polyesters include, but are not limited to, glycerol triacetate, acetylated-monoglyceride, diethyl phthalate, triethyl citrate, tributyl citrate, acetyl triethyl citrate, acetyl tributyl citrate.

Examples of suitable dimethicone copolyols include, but are not limited to, PEG-12 dimethicone, PEG/PPG-18/18 dimethicone, and PPG-12 dimethicone.

Particularly preferred examples of plasticizers include glycerin, ethylene glycol, polyethylene glycol, propylene glycol, and mixtures thereof. Most preferred plasticizer is glycerin.

### 4. Additional Ingredients

In addition to the above-described ingredients, e.g., the water-soluble polymer, the surfactant(s) and the plasticizer, the solid sheet of the present invention may comprise one or more additional ingredients, depending on its intended application. Such one or more additional ingredients may be selected from the group consisting of fabric care actives, dishwashing actives, hard surface cleaning actives, beauty and/or skin care actives, personal cleansing actives, hair care actives, oral care actives, feminine care actives, baby care actives, a bittering agent and any combinations thereof. In a preferred embodiment, the solid sheet of the present invention may comprise a bittering agent.

The solid sheet of the present invention may further comprise other optional ingredients that are known for use or otherwise useful in compositions, provided that such optional materials are compatible with the selected essential materials described herein, or do not otherwise unduly impair product performance.

Non-limiting examples of product type embodiments that can be formed by the solid sheet of the present invention include laundry detergent products, fabric softening products, hand cleansing products, hair shampoo or other hair treatment products, body cleansing products, shaving preparation products, dish cleaning products, personal care substrates containing pharmaceutical or other skin care actives, moisturizing products, sunscreen products, beauty or skin care products, deodorizing products, oral care products, feminine cleansing products, baby care products, fragrance-containing products, and so forth.

The solid sheet article of the present invention may further comprise other optional ingredients that are known for use or otherwise useful in compositions, provided that such optional materials are compatible with the selected essential materials described herein, or do not otherwise unduly impair product performance.

Non-limiting examples of product type embodiments that can be formed by the solid sheet article of the present invention include laundry detergent products, fabric softening products, hand cleansing products, hair shampoo or other hair treatment products, body cleansing products, shaving preparation products, dish cleaning products, personal care substrates containing pharmaceutical or other skin care actives, moisturizing products, sunscreen products, beauty or skin care products, deodorizing products, oral care products, feminine cleansing products, baby care products, fragrance-containing products, and so forth.

### Test 1: Scanning Electron Microscopic (SEM) Method for Determining Surface Average Pore Diameter of the Sheet Article

A Hitachi TM3000 Tabletop Microscope (S/N: 123104-04) is used to acquire SEM micrographs of samples. Samples of the solid sheet articles of the present invention are approximately 1 cm×1 cm in area and cut from larger sheets. Images are collected at a magnification of 50×, and the unit is operated at 15 kV. A minimum of 5 micrograph images are collected from randomly chosen locations across each sample, resulting in a total analyzed area of approximately 43.0 mm² across which the average pore diameter is estimated.

The SEM micrographs are then firstly processed using the image analysis toolbox in Matlab. Where required, the images are converted to grayscale. For a given image, a histogram of the intensity values of every single pixel is generated using the ‘imhist’ Matlab function. Typically, from such a histogram, two separate distributions are obvious, corresponding to pixels of the brighter sheet surface and pixels of the darker regions within the pores. A threshold value is chosen, corresponding to an intensity value between the peak value of these two distributions. All pixels having an intensity value lower than this threshold value are then set to an intensity value of 0, while pixels having an intensity value higher are set to 1, thus producing a binary black and white image. The binary image is then analyzed using ImageJ (https://imagej.nih.gov, version 1.52a), to examine both the pore area fraction and pore size distribution. The scale bar of each image is used to provide a pixel/mm scaling factor. For the analysis, the automatic thresholding and the analyze particles functions are used to isolate each pore. Output from the analyze function includes the area fraction for the overall image and the pore area and pore perimeter for each individual pore detected.

Average Pore Diameter is defined as D_{A}50: 50% of the total pore area is comprised of pores having equal or smaller hydraulic diameters than the D_{A}50 average diameter.

$$Hydraulic diameter = ‘ 4 * Pore area ( {m}^{2} ) / Pore perimeter ( m ) ’ .$$

It is an equivalent diameter calculated to account for the pores not all being circular.

### Test 2: Micro-Computed Tomographic (μCT) Method for Determining Overall or Regional Average Pore Size and Average Cell Wall Thickness of the Open Cell Foams (OCF)

Porosity is the ratio between void-space to the total space occupied by the OCF. Porosity can be calculated from μCT scans by segmenting the void space via thresholding and determining the ratio of void voxels to total voxels. Similarly, solid volume fraction (SVF) is the ratio between solid-space to the total space, and SVF can be calculated as the ratio of occupied voxels to total voxels. Both Porosity and SVF are average scalar-values that do not provide structural information, such as, pore size distribution in the height-direction of the OCF, or the average cell wall thickness of OCF struts.

To characterize the 3D structure of the OCFs, samples are imaged using a μCT scanning instrument capable of acquiring a dataset at high isotropic spatial resolution. Here is GE phoenix v|tome|×m with the follow settings: 240 kv micro focus tube, 180 kilo-volt and 120 micro-ampere, 500 projections images; 500 micro-second and 5 averages, a voxel size less than 10 micrometer per pixel. After scanning, the projection images are reconstructed into the 3D image and converted to 8-bit format stack of two dimensional images in the height-direction (or Z-direction) for analysis.

The stack of those images was used to estimate the change in sphere diameter from slice to slice as a function of OCF depth. It is, firstly, differentiate the wall and pore via threshold, and then run the “Local Thickness” script in ImageJ (see Robert Dougherty and Karl-Heinz Kunzelmann, Microscopy & Microanalysis, August 2007, 13 (S02)) to obtain the pore diameter.

To obtain a sample for measurement, lay a single layer out flat and cut a piece. During sampling, folds, wrinkles, and tears are avoided. Likewise, possibility of distortion and compression are minimized. A ring holder is used to support the cut sample so that direct pressure to its upper and bottom surfaces can be avoided.

The “Local Thickness” script gives a calculate result of sphere diameter from slice to slice. For Overall Average Pore Diameter (μm), it is the average of all the diameter number. For Top Average Pore Diameter (μm), Middle Average Pore Diameter (μm) and Bottom Average Pore Diameter (μm), the image stack is divided into three equal portions from top to bottom, and then calculate the average of each part.

### Test 3: Percent Open Cell Content of the Sheet Article

The Percent Open Cell Content is measured via gas pycnometry. Gas pycnometry is a common analytical technique that uses a gas displacement method to measure volume accurately. Inert gases, such as helium or nitrogen, are used as the displacement medium. A sample of the solid sheet article of the present invention is sealed in the instrument compartment of known volume, the appropriate inert gas is admitted, and then expanded into another precision internal volume. The pressure before and after expansion is measured and used to compute the sample article volume.

ASTM Standard Test Method D2856 provides a procedure for determining the percentage of open cells using an older model of an air comparison pycnometer. This device is no longer manufactured. However, one can determine the percentage of open cells conveniently and with precision by performing a test which uses Micromcritics' AccuPyc Pycnometer. The ASTM procedure D2856 describes 5 methods (A, B, C, D, and E) for determining the percent of open cells of foam materials. For these experiments, the samples can be analyzed using an Accupyc 1340 using nitrogen gas with the ASTM foampyc software. Method C of the ASTM procedure is to be used to calculate to percent open cells. This method simply compares the geometric volume as determined using calipers and standard volume calculations to the open cell volume as measured by the Accupyc, according to the following equation:

Open cell percentage=Open cell volume of sample/Geometric volume of sample*100

It is recommended that these measurements be conducted by Micromeretics Analytical Services, Inc. (One Micromeritics Dr, Suite 200, Norcross, GA 30093). More information on this technique is available on the Micromeretics Analytical Services web sites (www.particletesting.com or www.micromcritics.com), or published in “Analytical Methods in Fine particle Technology” by Clyde Orr and Paul Webb.

### Test 4: Final Moisture Content of the Sheet Article

Final moisture content of the solid sheet article of the present invention is obtained by using a Mettler Toledo HX204 Moisture Analyzer (S/N B706673091). A minimum of 1 g of the dried sheet article is placed on the measuring tray. The standard program is then executed, with additional program settings of 10 minutes analysis time and a temperature of 110° C.

### Test 5: Thickness of the Sheet Article

Thickness of the flexible, porous, dissolvable solid sheet article of the present invention is obtained by using a micrometer or thickness gage, such as the Mitutoyo Corporation Digital Disk Stand Micrometer Model Number IDS-1012E (Mitutoyo Corporation, 965 Corporate Blvd, Aurora, IL, USA 60504). The micrometer has a 1-inch diameter platen weighing about 32 grams, which measures thickness at an application pressure of about 0.09 psi (6.32 gm/cm²).

The thickness of the flexible, porous, dissolvable solid sheet article is measured by raising the platen, placing a section of the sheet article on the stand beneath the platen, carefully lowering the platen to contact the sheet article, releasing the platen, and measuring the thickness of the sheet article in millimeters on the digital readout. The sheet article should be fully extended to all edges of the platen to make sure thickness is measured at the lowest possible surface pressure, except for the case of more rigid substrates which are not flat.

### Test 6: Basis Weight of the Sheet Article

Basis Weight of the flexible, porous, dissolvable solid sheet article of the present invention is calculated as the weight of the sheet article per area thereof (grams/m²). The area is calculated as the projected area onto a flat surface perpendicular to the outer edges of the sheet article. The solid sheet articles of the present invention are cut into sample squares of 10 cm×10 cm, so the area is known. Each of such sample squares is then weighed, and the resulting weight is then divided by the known area of 100 cm² to determine the corresponding basis weight.

For an article of an irregular shape, if it is a flat object, the area is thus computed based on the area enclosed within the outer perimeter of such object. For a spherical object, the area is thus computed based on the average diameter as 3.14×(diameter/2)². For a cylindrical object, the area is thus computed based on the average diameter and average length as diameter×length. For an irregularly shaped three-dimensional object, the area is computed based on the side with the largest outer dimensions projected onto a flat surface oriented perpendicularly to this side. This can be accomplished by carefully tracing the outer dimensions of the object onto a piece of graph paper with a pencil and then computing the area by approximate counting of the squares and multiplying by the known area of the squares or by taking a picture of the traced area (shaded-in for contrast) including a scale and using image analysis techniques.

### Test 7: Density of the Sheet Article

Density of the flexible, porous, dissolvable solid sheet article of the present invention is determined by the equation: Calculated Density=Basis Weight of porous solid/(Porous Solid Thickness×1,000). The Basis Weight and Thickness of the dissolvable porous solid are determined in accordance with the methodologies described hereinabove.

### Test 8: Specific Surface Area of the Sheet Article

The Specific Surface Area of the flexible, porous, dissolvable solid sheet article is measured via a gas adsorption technique. Surface Area is a measure of the exposed surface of a solid sample on the molecular scale. The BET (Brunauer, Emmet, and Teller) theory is the most popular model used to determine the surface area and is based upon gas adsorption isotherms. Gas Adsorption uses physical adsorption and capillary condensation to measure a gas adsorption isotherm. The technique is summarized by the following steps; a sample is placed in a sample tube and is heated under vacuum or flowing gas to remove contamination on the surface of the sample. The sample weight is obtained by subtracting the empty sample tube weight from the combined weight of the degassed sample and the sample tube. The sample tube is then placed on the analysis port and the analysis is started. The first step in the analysis process is to evacuate the sample tube, followed by a measurement of the free space volume in the sample tube using helium gas at liquid nitrogen temperatures. The sample is then evacuated a second time to remove the helium gas. The instrument then begins collecting the adsorption isotherm by dosing krypton gas at user specified intervals until the requested pressure measurements are achieved. Samples may then analyzed using an ASAP 2420 with krypton gas adsorption. It is recommended that these measurements be conducted by Micromeretics Analytical Services, Inc. (One Micromeritics Dr, Suite 200, Norcross, GA 30093). More information on this technique is available on the Micromeretics Analytical Services web sites (www.particletesting.com or www.micromeritics.com), or published in a book, “Analytical Methods in Fine Particle Technology”, by Clyde Orr and Paul Webb.

### Test 9: Dissolution Rate

Firstly, the solid sheets are stored under ambient relative humidity of 50±2% and ambient temperature of 23±1° C. for 24 hours (i.e., a conditioning step). Following the initial conditioning step described above, 25 mm diameter discs are firstly cut from the large solid sheet using a 25 mm hollow hole punch. The required number of foam discs is set such that the total mass of all foam discs is no less than 0.1 g.

The required number of foam discs are then stacked in a head to toe orientation and placed inside an Omnifit™ EZ chromatography column (006EZ-25-10-AF) having 25 mm inner diameter, 100 m length and an adjustable, removable endpiece. The stack of foam discs is placed inside the column such that the direction of flow through the column is perpendicular to the top surface of the foam discs. Once placed inside the column, the endpiece is inserted into the column and adjusted until the perpendicular distance between the two inner frits is equal to the thickness of the stack of foam discs.

Masterflex silicone tubing (MFLEX SILICONE #25 25′) and a Masterflex peristaltic pump (MFLX L/S 1CH 300R 115/230 13124) are used to control the flow of water through the column. The system flow rate is calibrated by flowing water through the pump, tubing and an empty column at different pump RPM settings and recording the volume of water collected over a defined period of time. For all experiments a flow rate of 5 litres per hour was utilized.

The inlet and outlet tubing are both placed inside a 1 litre beaker containing 500 ml of deionised water at ambient temperature. The beaker is placed on a magnetic stirrer plate, and a magnetic stirrer bar having length 23 mm and thickness 10 mm is placed in the beaker, and the stirrer rotation speed is set to 300 rpm. A Mettler Toledo S230 conductivity meter is calibrated to 1413 μS/cm and the probe placed in the beaker of water.

The flow of water through the system is started. Once the first drops of water can be visibly seen inside the column and in contact with the foam, the data recording function of the conductivity meter is started. Data is recorded for at least 20 minutes.

In order to estimate the time required to reach a 90 or 95% percentage dissolution of the foam, a calibration curve is firstly generated where layers of the foam discs are dropped one a time into a stirred beaker of 500 ml deionised water. The mass of each individual foam disc, and the conductivity after 5 minutes are both recorded. This process is repeated for up to 5 discs total. A linear function is fitted to the data, which is then used to estimate the maximum conductivity in each dissolution experiment based on the total mass of the foam discs placed in the column. The percentage dissolution is then calculated as

$$\% Dissolution = Experimentally measured conductivity / Maximum conductivity * 100$$

The time required to achieve 90 or 95 percentage dissolution is then found from this calculated data. The calibration procedure is repeated for each formula tested.

### Test 10: Bubble Size

The bubble size of aerated pre-mixture is measured as follows:

Rectangular glass cover slides, having a width and a length of 2 cm and a thickness of 1 mm are firstly glued onto a glass slide having a width of 6 cm and a length of 2 cm, such that a cavity having a thickness of 1 mm, a length of 2 cm and a width of slightly less than 2 cm is located in the center of the glass slide. The width of the cavity must be kept at less than 2 cm so that an additional cover slide can be placed on top of the cavity.

To capture the image for bubble size analysis, the aerated liquid foam is deposited into the cavity using a spatula and another cover slide placed on top and pressed down gently, in order to reduce the thickness of the liquid to 1 mm.

A SMZ-T4 Chongqing Optec microscope and RZIMAGE MicroUL300 digital camera were used to capture the images. The glass slide was placed onto the backlit area of the microscope, and the magnification adjusted such that the image area was no less than 16 mm². An additional image was taken with a transparent ruler placed in the image area, such that the graduated lines could be seen and used to determine the pixel to distance ratio.

The bubble sizes were calculated using the ‘imfindcircles’ function in the Image Analysis Toolbox of the Matlab 2017b software. For each image, the function was called four times, for pixel size ranges of 21 to 40, 41 to 50, 51 to 100 and 101 to 200, respectively, where 20 pixels corresponds to an approximate length of 60 micron. The sensitivity parameter was set to 0.95. The bubble radii estimated from each call of the function were combined to generate a single distribution, and the radii converted to microns using the calibration image generated with the transparent ruler.

Test 11: Delamination of multilayer solid sheet articles

Delamination of multilayer solid sheet articles containing a loading composition is assessed by using a drop test. The solid sheets used in this test prepared according to the present disclosure are firstly conditioned by placing them in a temperature and humidity controlled room, with temperature and humidity controlled in the range 23 to 24.5° C. and 41 to 45% relative humidity respectively, for a minimum of 1 hour. The sheets are laid out individually and not stacked upon one another.

10×10 cm square samples of foam sheets are then cut out from the larger sheets by utilizing a paper guillotine. All four edges of the 10×10 cm square are cut by utilizing the paper guillotine. None of the existing edges of the larger sheet stack are used as edges of the smaller 10×10 cm square.

The loading composition is then added to the center of one of the 10×10 cm square samples, and a second 10×10 cm sample then placed on top of the first sample, such that the two sheet layers are orientated in toe-to-toe configuration. No excessive pressure is applied to the sheet stack during the testing, wherein the excessive pressure is defined as any pressure resulting in a 0.05 mm or greater thickness change of the sheet. The 10×10 cm sample is further cut by using the paper guillotine to a smaller 5×5 cm sample, in which the loading composition is remained in the center of the 5×5 cm sample.

The drop test is carried out as follows. A plastic thumb forceps is utilized to place the test sample (i.e. 5×5 cm sample) 1.0 meter above a solid surface such as the floor or a tabletop. The sample is orientated such that one of the edges exposed by cutting is parallel to the solid surface. The sample is then released and allowed to fall. After dropping to the solid surface, the edges of the two sheet layers may separate due to the presence of the loading composition, resulting in leakage of the loading composition. As such, the percentage of adhesion can be used as a ‘leakage score’ to characterize the leakage degree of the loading composition. The ‘leakage score’ ranging from 0 to 5 is assigned to each sample, according to the following criteria.
0—Less than 5% of the two sheet layer edges are in contact with one another
1—Exactly 5% or between 5 to 25% of the two sheet layer edges are adhered to one another
2—Exactly 25% or between 25% to 50% of the two sheet layer edges are adhered to one another
3—Exactly 50% or between 50% to 75% of the two sheet layer edges are adhered to one another
4—Exactly 75% or between 75% to 95% of the two sheet layer edges are adhered to one another
5—Exactly 95% or greater than 95% of the two sheet layer edges are adhered to one another

For each sample edge, apparent separation between adjacent sheet layers is identified by naked eye observation and the length of the apparent edge separation is measured by using a ruler. The percentage of adhesion is then calculated as follows: Length of apparent edge separation summed across the four edges (centimeters)/the sum of the four edge lengths (centimeters). A minimum of 3 repeat measurements for drop test is carried out for each formulation.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, i.e., wt/wt percentages, unless otherwise specified.

### Example 1: Improved Performance of the Solid Sheet Article Containing a Preferred Ratio of AAS to AS

Dissolvable unit dose articles containing two flexible, porous, solid sheets were prepared as follows. Particularly, large flexible, porous, solid sheets (with minimum area 1.0×1.0 m) were prepared according to the method in the present disclosure.

Specifically, a wet pre-mixture (i.e., a slurry) containing the ingredients of the solid sheet shown in the following Table 1 and additional water was prepared, to result in a total solids content of around 38% by weight (i.e., the total water content in the slurry is around 62% by weight).

TABLE 1

| | Sheet A | | Sheet B | | Sheet C | | Sheet D | | Sheet E | |
|---|---|---|---|---|---|---|---|---|---|---|
| | (Wet) | (Wet) | (Wet) | (Dry) | (Wet) | (Dry) | (Wet) | (Dry) | (Wet) | (Dry) |
| Materials: | w/w % | w/w % | w/w % | w/w % | w/w % | w/w % | w/w % | w/w % | w/w % | w/w % |
| Polyvinyl alcohol (High | 6.1 | 15 | 6.1 | 15 | 4 | 10 | 4 | 10 | 6.1 | 15 |
| Molecular Weight)¹ | | | | | | | | | | |
| Polyvinyl alcohol (Low | 4.9 | 12 | 4.9 | 12 | 8.1 | 20 | 8.1 | 20 | 4.9 | 12 |
| Molecular Weight)² | | | | | | | | | | |
| Glycerin | 2.4 | 6 | 2.4 | 6 | 1.6 | 4.0 | 1.6 | 4.0 | 2.4 | 6 |
| Sodium Lauryl Sulfate | 20.2 | 50 | 0 | 0 | 15.0 | 37.0 | 4.0 | 10.0 | 10.1 | 25 |
| Sodium Laureth 3 Sulfate | 0 | 0 | 20.2 | 50 | 4.0 | 10.0 | 15.0 | 37.0 | 10.1 | 25 |
| Polyalkylene imine | 2 | 5 | 2 | 5 | 2 | 5 | 2 | 5 | 2 | 5 |
| polymer A³ | | | | | | | | | | |
| Soap | 0 | 0 | 0 | 0 | 0 | 0 | 0 | 0 | 0 | 0 |
| Zeolite | 0.4 | 1 | 0.4 | 1 | 1.2 | 3 | 1.2 | 3 | 0.4 | 1 |
| Chelant | 0.8 | 2 | 0.8 | 2 | 1.2 | 3 | 1.2 | 3 | 0.8 | 2 |
| Anti-oxidant | 0.8 | 2 | 0.8 | 2 | 0.8 | 2 | 0.8 | 2 | 0.8 | 2 |
| Perfume Microcapsule | 0.4 | 1 | 0.4 | 1 | 0 | 0 | 0 | 0 | 0.4 | 1 |
| Water | 62 | 6 | 62 | 6 | 62 | 6 | 62 | 6 | 62 | 6 |

| | Sheet F | | Sheet G | | Sheet H | | Sheet I | |
|---|---|---|---|---|---|---|---|---|
| | (Wet) | (Dry) | (Wet) | (Dry) | (Wet) | (Dry) | (Wet) | (Dry) |
| Materials: | w/w % | w/w % | w/w % | w/w % | w/w % | w/w % | w/w % | w/w % |
| Polyvinyl alcohol (High | 4 | 10 | 4 | 10 | 4 | 10 | 3.7 | 10 |
| Molecular Weight)¹ | | | | | | | | |
| Polyvinyl alcohol (Low | 8.1 | 20 | 8.1 | 20 | 8.1 | 20 | 7.4 | 20 |
| Molecular Weight)² | | | | | | | | |
| Glycerin | 2.4 | 6 | 2.4 | 6 | 2.4 | 6 | 1.5 | 4.0 |
| Sodium Lauryl Sulfate | 12.1 | 30 | 6.1 | 15 | 9.1 | 22.5 | 9.3 | 25.0 |
| Sodium Laureth 3 Sulfate | 6.1 | 15 | 12.1 | 30 | 9.1 | 22.5 | 9.3 | 25.0 |
| Polyalkylene imine | 2 | 5 | 2 | 5 | 2 | 5 | 0 | 0 |
| polymer A³ | | | | | | | | |
| Soap | 0 | 0 | 0 | 0 | 0 | 0 | 0.7 | 2 |
| Zeolite | 1.2 | 3 | 1.2 | 3 | 0.4 | 3 | 0.7 | 2 |
| Chelant | 0.8 | 2 | 0.8 | 2 | 0.8 | 2 | 1.1 | 3 |
| Anti-oxidant | 0.8 | 2 | 0.8 | 2 | 0.8 | 2 | 0.7 | 2 |
| Perfume Microcapsule | 0.4 | 1 | 0.4 | 1 | 0.4 | 1 | 0.4 | 1 |
| Water | 62 | 6 | 62 | 6 | 62 | 6 | 65 | 6 |

¹Molecular weight 85,000, Degree of Hydrolysis 87%.
²Molecular weight 25,000, Degree of Hydrolysis 87%
³Polyethylenimine polymer with 24 EO and 16 PO per —NH with MW 600 from BASF

The slurry so formed was then aerated and dried in a belt drier by using parameters as shown below to form a solid sheet (i.e., Sheets A to I). Particularly, the belt drier comprises three heating zones in which the three heating zones are configured to simultaneously heat the top and bottom sides of said formed sheet independently at a first top heating temperature (Tₜ₁) and a first bottom heating temperature (T_{b1}) for a first heating duration of from 0.01 minutes to 20 minutes in the Heating Zone 1, heat the top and bottom sides of said formed sheet independently at a first top heating temperature (Tₜ₂) and a first bottom heating temperature (T_{b2}) for a second heating duration of from 0.01 minutes to 20 minutes in the Heating Zone 2, and heat the top and bottom sides of said formed sheet independently at a first top heating temperature (Tₜ₃) and a first bottom heating temperature (T_{b3}) for a third heating duration of from 0.01 minutes to 20 minutes in the Heating Zone 3. The parameters are shown in Table 2 below.

TABLE 2

| Drying method/Temperature | Parameters |
|---|---|
| Belt drier (Step-wise heating) | Slurry Temperature before and |
| Bottom 120-80° C./Top 100-140° C. | during aeration: 60-70° C. |
| Sequential Bottom Zones 1 to 3: | Mixing head speed setting for |
| 120° C. 120° C. 80° C. | aerator: 300 |
| Sequential Top Zones 1 to 3: | Air flow rate setting for |
| 100° C. 120° C. 140° C. | aerator: 100 |
| | Slurry Temperature before drying: |
| | 60-70° C. |
| | Drying linear speed: 0.5 to |
| | 1.0 m/min |
| | Heating zone area |
| | (each): ~3.4 m × ~0.4 m |
| | (length × width) |

The results of manufacturing the flexible, porous, solid sheets as shown in Table 1 (i.e. Sheets A to I) are shown in Table 3. It indicates that the manufacturing can be successful only when the ratio of AAS to AS is within a preferred range.

TABLE 3

| | Sheet A | Sheet B | Sheet C | Sheet D | Sheet E | Sheet F | Sheet G | Sheet H | Sheet I |
|---|---|---|---|---|---|---|---|---|---|
| Ratio of | AS | AAS | 0.3 | 3.7 | 1.0 | 2.0 | 0.5 | 1.0 | 1.0 |
| AAS/AS | alone | alone | | | | | | | |
| Wet Pre- | 400 | 350 | 400 | 400 | 400 | 350 | 350 | 350 | 320 |
| Mixture | | | | | | | | | |
| Density (g/cm³) | | | | | | | | | |
| Dry Sheet | 114 | 314 | 133 | 230 | 156 | 163 | 161 | 150 | 150 |
| Density (g/cm³) | | | | | | | | | |
| Thickness (mm) | 2.5 | 1.0 | 1.7 | 1.0 | 1.8 | 1.9 | 1.8 | 1.6 | 2.2 |
| Basis weight | 285 | 314 | 220 | 230 | 280 | 310 | 290 | 240 | 328 |
| (g/m²) | | | | | | | | | |
| Collapse % | −41.8% | 54.9% | −21.3% | 29.7% | −4.0% | 13.3% | 12.2% | 5.7% | 13.8% |
| Manufacturing | Failure | Failure | Failure | Failure | Success | Success | Success | Success | Success |
| Results | | | | | | | | | |

Particularly, Collapse % is calculated according to the following equation:

$$Collapse \% = 1 - \frac{{ρ}_{L} \frac{( 1 - {w}_{L} )}{( 1 - {w}_{D} )}}{{ρ}_{D}}$$

Where ρ_{L} is the density of the aerated wet pre-mixture, w_{L} is the water content of the wet pre-mixture, w_{D} is the water content of the dry sheet and pp is the density of the dry sheet. A collapse of greater than 20% is undesirable and as such determined as a failure because it indicates the porous structure has significantly collapsed during drying, which may result in poor dissolution of the sheet in water. Similarly, a collapse of lower than-20% is undesirable as well and as such determined as a failure because it indicates puffing has occurred during drying, which may result in easy tearing of the sheet during handling.

### Example 2: Improved Delamination Performance of the Multilayer Solid Sheet Articles

Further, the delamination performance of the unit dose articles was tested in accordance with Test 11: Delamination of multilayer solid sheet articles. Similarly as in Example 1, Sheets H and I were prepared according to the method of the present disclosure. Subsequently, unit dose articles were prepared by loading a loading composition in a form of powder containing LAS, citric acid, silica and perfume within two sheets at a weight ratio of 11 g:5 g (sheets: loading composition) as prepared above by using the conversion method according to the present disclosure. Particularly, a first sheet and a second sheet were respectively fed on a belt conveyor sequentially. The loading composition was loaded onto the first sheet, and spread evenly between the first and second sheets. Finally, the stack of sheets passed through the edge sealing unit comprising an edge sealing roller as shown in FIG. 3 which comprises a tooling as shown in FIG. 4 (Temperature: 150° C.; Pressure: 2000 psi) so that the stack of sheets was cut into unit dose articles with sealed edge. Then, the unit dose articles as prepared were tested to determine if the sealing was successful, i.e., to see if a significant separation between layers (i.e., delamination) or leakage is present. Particularly, the unit dose articles were stored at 25° C. and 40% RH for 48 hours and then checked to see if the edge was still sealed or any significant leakage happened. If the edge was still sealed and no significant leakage happened, the sealing was successful. If the edge was not sealed any more, any separation between layers happened, or any significant leakage happened, the sealing was not successful. The inventors surprisingly found that the performance of delamination is great for both Sheets H and I because no separation was identified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A flexible, porous, dissolvable solid sheet article comprising a plurality of water-soluble sheets arranged in a stack, wherein each of the water-soluble sheets comprises a water-soluble polymer, a C₆-C₂₀ linear or branched alkylalkoxy sulfates (AAS) having a weight average degree of alkoxylation ranging from 0.5 to 10, and a C₆-C₂₀ linear or branched alkyl sulfates (AS),
wherein said solid sheet article is characterized by: (i) a Percent Open Cell Content of from 80% to 100%; and (ii) an Overall Average Pore Size of from 100 μm to 2000 μm;
wherein the weight ratio of said AAS over said AS is from 0.4 to 3.

2. The flexible, porous, dissolvable solid sheet article of claim 1, wherein the weight ratio of said AAS over said AS is from 0.5 to 2.

3. The flexible, porous, dissolvable solid sheet article of claim 1, wherein each of the water-soluble sheets comprises less than 5% of a non-ionic surfactant by total weight of the water-soluble sheet.

4. The flexible, porous, dissolvable solid sheet article of claim 1, wherein said water-soluble polymer is selected from the group consisting of polyvinyl alcohols, polyvinylpyrrolidones, polyalkylene oxides, starch and starch derivatives, pullulan, gelatin, hydroxypropylmethylcelluloses, methycelluloses, carboxymethycelluloses, and any combinations thereof; and
wherein said water-soluble polymer has a weight average molecular weight of from 5,000 to 400,000 Daltons.

5. The flexible, porous, dissolvable solid sheet article of claim 4, wherein the water-soluble polymer comprises a polyvinyl alcohol; and
wherein said solid sheet article comprises from 1% to 60% of said polyvinyl alcohol by total weight of said solid sheet article.

6. The flexible, porous, dissolvable solid sheet article of claim 1, wherein each of the water-soluble sheets comprises from 1% to 60% of total surfactants by total weight of said solid sheet article.

7. The flexible, porous, dissolvable solid sheet article of claim 1, wherein each of the water-soluble sheets comprises an additional surfactant, a perfume, a chelant, a polymer, a solvent, an antioxidant, or any combinations thereof,
wherein said additional surfactant is selected from the group consisting of: a C₆-C₂₀ linear alkylbenzene sulfonate (LAS), a fatty acid, alkylamphoacetates and any combinations thereof;
and/or wherein said perfume is selected from the group consisting of: free perfumes, perfume microcapsules, and any combinations thereof; and/or
wherein said chelant is selected from the group consisting of diamine-N,N′-dipolyacid, monoamine monoamide-N,N′-dipolyacid, and N,N′-bis(2-hydroxybenzyl)ethylenediamine-N,N′-diacetic acid chelants, carboxylic acids, phosphonic acids and polyphosphoric, their salts and derivatives; and/or
wherein said polymer is selected from the group consisting of polyalkylene imine polymer, polyalkylene oxide polymer and any combinations thereof; and/or
wherein said solvent is selected from the group consisting of glycerol, propylene glycol, 1,3-propanediol, diethylene glycol, dipropylene glycol, ethanolamine, ethanol, water and any combinations thereof; and/or
wherein said antioxidant is selected from the group consisting of C₁-C₂₂ linear alkyl esters of 3,5-bis(1,1-dimethylethyl)-4-hydroxy-benzenepropanoic acid and mixtures thereof.

8. The flexible, porous, dissolvable solid sheet article of claim 1, wherein said solid sheet article is characterized by:
a Percent Open Cell Content of from 85% to 100%; and/or
an Overall Average Pore Size of from 150 μm to 1000 μm; and/or
an Average Cell Wall Thickness of from 5 μm to 200 μm; and/or
a final moisture content of from 0.5% to 25% by weight of said solid sheet article; and/or
a thickness of each sheet being from 0.5 mm to 4 mm; and/or
a basis weight of from 50 grams/m² to 500 grams/m²; and/or
a density of from 0.05 grams/cm³ to 0.5 grams/cm³; and/or
a Specific Surface Area of from 0.03 m²/g to 0.25 m²/g.

9. The flexible, porous, dissolvable solid sheet article of claim 1, wherein the flexible, porous, dissolvable solid sheet article further comprises a loading composition in a form of paste or powders between adjacent two sheets.

10. The flexible, porous, dissolvable solid sheet article of claim 9, wherein the loading composition is in a form of non-aqueous paste and comprises a non-aqueous liquid carrier and solid particles, wherein said non-aqueous paste comprises:
from 1% to 99% of a non-aqueous liquid carrier by total weight of said non-aqueous paste;
and/or from 1% to 99% of solid particles by total weight of said non-aqueous paste.

11. The flexible, porous, dissolvable solid sheet article of claim 10, wherein said non-aqueous liquid carrier is selected from the group consisting of polyethylene glycol, polypropylene glycol, silicone, fatty acid, perfume oil, a non-ionic surfactant, an organic solvent and any combinations thereof, wherein said non-aqueous liquid carrier comprises a non-ionic surfactant that is selected from the group consisting of C₆-C₂₀ linear or branched alkylalkoxylated alcohols (AA) having a weight average degree of alkoxylation ranging from 5 to 15; and/or
wherein said solid particles comprise an oxidative dye compound, a pH modifier and/or a buffering agent, a radical scavenger, a chelant, a warming active, a color indicator, an anionic surfactant, an enzyme, a bleaching agent, an effervescent system and any combinations thereof,
wherein said solid particles comprises C₆-C₂₀ linear alkylbenzene sulphonate (LAS) surfactant, percarbonate salts, perborate salts, persulfate salts, tetraacetylethylenediamine (TAED), oxybenzene sulphonates, caprolactams, or any combinations thereof.

12. The flexible, porous, dissolvable solid sheet article of claim 9, wherein the loading composition is in a form of powders which are characterized by a bulk density of from 250 g/l to 500 g/l,
wherein the powders are characterized by a mean particle size of from about 200 to about 600 microns.

13. The flexible, porous, dissolvable solid sheet article of claim 12, wherein said powders comprises an anionic surfactant which is selected from the group consisting of C₆-C₂₀ linear alkylbenzene sulfonate (LAS), a C₆-C₂₀ linear or branched alkylalkoxy sulfates (AAS) having a weight average degree of alkoxylation ranging from 0.5 to 10, a C₆-C₂₀ linear or branched alkyl sulfates (AS) and any combinations thereof.

14. The flexible, porous, dissolvable solid sheet article of claim 1, wherein the C₆-C₂₀ linear or branched alkylalkoxy sulfates (AAS) having a weight average degree of alkoxylation ranging from 0.5 to 10 is selected from the group consisting of linear or branched alkylethoxy sulfates (AES) having the respective formulae RO(C₂H₄O)ₓSO₃M, wherein R is alkyl or alkenyl of from about 6 to about 18 carbon atoms, x is from 1 to 10, and M is a water-soluble cation such as ammonium, sodium, potassium and triethanolamine; and/or
wherein the C₆-C₂₀ linear or branched alkyl sulfates (AS) is selected from the group consisting of alkyl sulfates having the respective formulae ROSO₃M, wherein R is alkyl or alkenyl of from about 6 to about 18 carbon atoms, and M is a water-soluble cation such as ammonium, sodium, potassium and triethanolamine.

15. The flexible, porous, dissolvable solid sheet article of claim 1, wherein the C₆-C₂₀ linear or branched alkylalkoxy sulfates (AAS) having a weight average degree of alkoxylation ranging from 0.5 to 10 is AES having the respective formulae RO(C₂H₄O)ₓSO₃M, wherein R is alkyl or alkenyl of from about 10 to about 14 carbon atoms, x is from 3 to 5, and M is a water-soluble cation such as ammonium, sodium, potassium and triethanolamine; and
wherein the C₆-C₂₀ linear or branched alkyl sulfates (AS) is selected from the group consisting of alkyl sulfates having the respective formulae ROSO₃M, wherein R is alkyl or alkenyl of from about 10 to about 14 carbon atoms, and M is a water-soluble cation such as ammonium, sodium, potassium and triethanolamine.
